(19) United States
(12) Plant Patent Application Publication
(10) Pub. No.: US 2025/0212707 P1
(43) Pub. Date: Jun. 26, 2025

(54) SELF-COMPATIBLE CACAO CLONES

(50) ;

(71) Applicant: Mars, Incorporated, McLean, VA (US)

(72) Inventors: Andrew WARD, Graceville (AU); Hussin Bin PURUNG, Makassar (ID); Smilja LAMBERT, McLean, VA (US)

(73) Assignee: Mars, Incorporated, McLean, VA (US)

(21) Appl. No.: 18/991,045

(22) Filed: Dec. 20, 2024

Related U.S. Application Data
(60) Provisional application No. 63/614,442, filed on Dec. 22, 2023.

(51) Int. Cl. A01H 6/00; A01H 5/08

(52) U.S. Cl. CPC A01H 6/00; A01H 5/08
USPC PLT/152

(57) ABSTRACT

A new and distinct variety of cacao plant selected from the group consisting of ‘ISC-1’. ‘ISC-2’, and ‘ISC-3’, particularly selected for its self-compatibility, is disclosed.

### CROSS-REFERENCE TO RELATED APPLICATION

This application claims the benefit of U.S. Provisional Application No. 63/614,442, filed Dec. 22, 2023, which is hereby incorporated by reference in its entirety.

Latin name: Botanical classification: Theobroma cacao L.

Varietal denomination: Variety denomination: the varietal denominations of the cacao varieties disclosed herein are ‘ISC-1’, ‘ISC-2’, and ‘ISC-3’.

### BACKGROUND OF THE INVENTION

Cacao beans are produced by cacao trees, which are found in warm, moist climates in areas about 20 degrees latitude north and south of the Equator. The cacao tree produces leaves, flowers and fruit throughout the year, and the ripe fruit or pod resembles a long cantaloupe, typically containing from about 20 to 40 almond-shaped cacao beans. Cacao butter and cacao solids are extracted from commercial cacao beans and processed to produce various cacao and chocolate products. Different varieties of cacao trees have varying cacao bean yield, resistance to disease, and levels of compounds that impact the flavor and nutritional value of cacao products. Cacao is an important crop that has been appreciated by humans for thousands of years.

Theobroma cacao L. trees predominantly outcross, with most trees being considered self-incompatible (also referred to as “SI”), meaning that other compatible clones must be planted with them to facilitate successful pollination and seed set. In some growing areas (e.g., Central America) many cultivated trees have undergone human-mediated selection for self-compatibility (also referred to as “SC”). However, in other growing areas (e.g., Indonesia), all of the cacao clones grown are self-incompatible, and the resulting need for other compatible clones to be planted with them is often not recognized by farmers. Monoclonal plantings are extremely common and severely curtail yields. There is a need for new self-compatible cacao clones to remove the risk of monoclonal plantings. Self-compatible cacao clones ensure that all pollination events have the potential to be successful and ensure high yields.

### SUMMARY OF THE INVENTION

In order to meet these needs, the present invention is directed to improved varieties of cacao plant. In particular, the invention relates to new and distinct varieties of cacao plant (Theobroma cacao L) that have been denominated as ‘ISC-1’, ‘ISC-2’, and ‘ISC-3’, respectively. Varieties ‘ISC-1’, ‘ISC-2’, and ‘ISC-3’ are self-compatible clones and are high yielding (more than 2.5 t/ha) and suitable for monoclonal plantations. When compared to the industry standard non-selfing clone ‘MCC02’, cumulative yields at year three of production of the self-compatible cacao clones of the present disclosure had a 3 t DB/ha advantage. Further, the disease tolerance of the self-compatible cacao clones appears to be good.

Each new cacao plant of the present invention is stable and reproduces true to type in successive generations of asexual reproduction.

Cacao plant variety ‘ISC-1’ originated from a controlled cross between the cacao female parent ‘MCC02’ (unpatented) and the cacao male parent ‘RUQ1347’ (unpatented). ‘ISC-1’ was asexually propagated via grafting onto rootstock in Pangkep, Indonesia. ‘ISC-1’ was particularly selected for its self-compatibility, in addition to the plant having high yield potential.

Cacao plant variety ‘ISC-2’ originated from a controlled cross between the cacao female parent ‘MCC02’ (unpatented) and the cacao male parent ‘S1’ (unpatented). ‘ISC-2’ was asexually propagated via grafting onto rootstock in Pangkep, Indonesia. ‘ISC-2’ was particularly selected for its self-compatibility, in addition to the plant having high yield potential.

Cacao plant variety ‘ISC-3’ originated from a controlled cross between the cacao female parent ‘MCC02’ (unpatented) and the cacao male parent ‘RUQ1347’ (unpatented). ‘ISC-3’ was asexually propagated via grafting onto rootstock in Pangkep, Indonesia. ‘ISC-3’ was particularly selected for its self-compatibility, in addition to the plant having high yield potential.

### BRIEF DESCRIPTION OF THE DRAWINGS

This new cacao plants are illustrated by the accompanying photographs. The colors shown are as true as can be reasonably obtained by conventional photographic procedures.

FIGS. 1A-1F illustrate leaves of variety ‘ISC-1’. FIG. 1A illustrates the length of a leaf blade of variety ‘ISC-1’. FIG. 1B illustrates the width of a leaf blade of variety ‘ISC-1’. FIG. 1C illustrates the shape of base of a leaf blade of variety ‘ISC-1’. FIG. 1D illustrates the intensity of green color of a leaf blade of variety ‘ISC-1’. FIG. 1E illustrates the shape of apex of a leaf blade of variety ‘ISC-1’. FIG. 1F illustrates the colors of young leaves of variety ‘ISC-1’ at the stages soon, early, and medium.

FIGS. 2A-2I illustrate flowers of variety ‘ISC-1’. FIG. 2A illustrates the anthocyanin coloration of pedicel of a flower of variety ‘ISC-1’. FIG. 2B illustrates the anthocyanin coloration of pedicel of a young flower of variety ‘ISC-1’. FIG. 2C illustrates the anthocyanin coloration of pedicel of a closed flower of variety ‘ISC-1’. FIG. 2D illustrates the anthocyanin coloration of pedicel of an open flower of variety ‘ISC-1’. FIG. 2E illustrates the length of sepal of a flower of variety ‘ISC-1’. FIG. 2F illustrates the width of sepal of a flower of variety ‘ISC-1’. FIG. 2G illustrates the anthocyanin coloration of sepal of a flower of variety ‘ISC-1’. FIG. 2H illustrates the color of ligula of a flower of variety ‘ISC-1’. FIG. 2I illustrates the anthocyanin coloration of staminode of a flower of variety ‘ISC-1’.

FIGS. 3A-3J illustrate fruit of variety ‘ISC-1’. FIG. 3A illustrates the shape of a fruit of variety ‘ISC-1’. FIG. 3B illustrates the basal constriction of a fruit of variety ‘ISC-1’. FIG. 3C illustrates the shape of apex of a fruit of variety ‘ISC-1’. FIG. 3D illustrates the fruit length of variety ‘ISC-1’. FIG. 3E illustrates the fruit diameter of variety ‘ISC-1’. FIG. 3F illustrates the fruit surface of variety ‘ISC-1’. FIG. 3G illustrates the fruit depth of grooves of variety ‘ISC-1’. FIG. 3H illustrates the fruit color of variety ‘ISC-1’. FIG. 3I illustrates the exocarp thickness of a fruit of variety ‘ISC-1’. FIG. 3J illustrates the color of pulp of a fruit of variety ‘ISC-1’.

FIGS. 4A-4F illustrate seeds of variety ‘ISC-1’. FIG. 4A illustrates the shape of a seed of variety ‘ISC-1’. FIG. 4B illustrates the shape in longitudinal section of a seed of variety ‘ISC-1’. FIG. 4C illustrates the seed length of variety ‘ISC-1’. FIG. 4D illustrates the seed width of variety ‘ISC-1’. FIG. 4E illustrates the seed thickness of variety ‘ISC-1’. FIG. 4F illustrates the seed cotyledon color of variety ‘ISC-1’.

FIGS. 5A-5F illustrate leaves of variety ‘ISC-2’. FIG. 5A illustrates the length of a leaf blade of variety ‘ISC-2’. FIG. 5B illustrates the width of a leaf blade of variety ‘ISC-2’. FIG. 5C illustrates the shape of base of a leaf blade of variety ‘ISC-2’. FIG. 5D illustrates the intensity of green color of a leaf blade of variety ‘ISC-2’. FIG. 5E illustrates the shape of apex of a leaf blade of variety ‘ISC-2’. FIG. 5F illustrates the colors of young leaves of variety ‘ISC-2’ at the stages soon, early, and medium.

FIGS. 6A-6H illustrate flowers of variety ‘ISC-2’. FIG. 6A illustrates the anthocyanin coloration of pedicel of a flower of variety ‘ISC-2’. FIG. 6B illustrates the anthocyanin coloration of pedicel of a closed flower of variety ‘ISC-2’. FIG. 6C illustrates the anthocyanin coloration of pedicel of an open flower of variety ‘ISC-2’. FIG. 6D illustrates the length of sepal of a flower of variety ‘ISC-2’. FIG. 6E illustrates the width of sepal of a flower of variety ‘ISC-2’. FIG. 6F illustrates the anthocyanin coloration of sepal of a flower of variety ‘ISC-2’. FIG. 6G illustrates the color of ligula of a flower of variety ‘ISC-2’. FIG. 6H illustrates the anthocyanin coloration of staminode of a flower of variety ‘ISC-2’.

FIGS. 7A-7J illustrate fruit of variety ‘ISC-2’. FIG. 7A illustrates the shape of a fruit of variety ‘ISC-2’. FIG. 7B illustrates the basal constriction of a fruit of variety ‘ISC-2’. FIG. 7C illustrates the shape of apex of a fruit of variety ‘ISC-2’. FIG. 7D illustrates the fruit length of variety ‘ISC-2’. FIG. 7E illustrates the fruit diameter of variety ‘ISC-2’. FIG. 7F illustrates the fruit surface of variety ‘ISC-2’. FIG. 7G illustrates the fruit depth of grooves of variety ‘ISC-2’. FIG. 7H illustrates the fruit color of variety ‘ISC-2’. FIG. 7I illustrates the exocarp thickness of a fruit of variety ‘ISC-2’. FIG. 7J illustrates the color of pulp of a fruit of variety ‘ISC-2’.

FIGS. 8A-8F illustrate seeds of variety ‘ISC-2’. FIG. 8A illustrates the shape of a seed of variety ‘ISC-2’. FIG. 8B illustrates the shape in longitudinal section of a seed of variety ‘ISC-2’. FIG. 8C illustrates the seed length of variety ‘ISC-2’. FIG. 8D illustrates the seed width of variety ‘ISC-2’. FIG. 8E illustrates the seed thickness of variety ‘ISC-2’. FIG. 8F illustrates the seed cotyledon color of variety ‘ISC-2’.

FIGS. 9A-9F illustrate leaves of variety ‘ISC-3’. FIG. 9A illustrates the length of a leaf blade of variety ‘ISC-3’. FIG. 9B illustrates the width of a leaf blade of variety ‘ISC-3’. FIG. 9C illustrates the shape of base of a leaf blade of variety ‘ISC-3’. FIG. 9D illustrates the intensity of green color of a leaf blade of variety ‘ISC-3’. FIG. 9E illustrates the shape of apex of a leaf blade of variety ‘ISC-3’. FIG. 9F illustrates the colors of young leaves of variety ‘ISC-3’ at the stages soon, early, and medium.

FIGS. 10A-10H illustrate flowers of variety ‘ISC-3’. FIG. 10A illustrates the anthocyanin coloration of pedicel of a flower of variety ‘ISC-3’. FIG. 10B illustrates the anthocyanin coloration of pedicel of an open flower of variety ‘ISC-3’. FIG. 10C illustrates the anthocyanin coloration of pedicel of a closed flower of variety ‘ISC-3’. FIG. 10D illustrates the length of sepal of a flower of variety ‘ISC-3’. FIG. 10E illustrates the width of sepal of a flower of variety ‘ISC-3’. FIG. 10F illustrates the anthocyanin coloration of sepal of a flower of variety ‘ISC-3’. FIG. 10G illustrates the color of ligula of a flower of Variety ‘ISC-3’. FIG. 10H illustrates the anthocyanin coloration of staminode of a flower of variety ‘ISC-3’.

FIGS. 11A-11J illustrate fruit of variety ‘ISC-3’. FIG. 11A illustrates the shape of a fruit of variety ‘ISC-3’. FIG. 11B illustrates the basal constriction of a fruit of variety ‘ISC-3’. FIG. 11C illustrates the shape of apex of a fruit of variety ‘ISC-3’. FIG. 11D illustrates the fruit length of variety ‘ISC-3’. FIG. 11E illustrates the fruit diameter of variety ‘ISC-3’. FIG. 11F illustrates the fruit surface of variety ‘ISC-3’. FIG. 11G illustrates the fruit depth of grooves of variety ‘ISC-3’. FIG. 11H illustrates the fruit color of variety ‘ISC-3’. FIG. 11I illustrates the exocarp thickness of a fruit of variety ‘ISC-3’. FIG. 11J illustrates the color of pulp of a fruit of variety ‘ISC-3’.

FIGS. 12A-12E illustrate seeds of variety ‘ISC-3’. FIG. 12A illustrates the shape in longitudinal section of a seed of variety ‘ISC-3’. FIG. 12B illustrates the seed length of variety ‘ISC-3’. FIG. 12C illustrates the seed width of Variety ‘ISC-3’. FIG. 12D illustrates the seed thickness of variety ‘ISC-3’. FIG. 12E illustrates the seed cotyledon color of variety ‘ISC-3’.

FIGS. 13A-13F illustrate leaves of comparison variety ‘MCC02’. FIG. 13A illustrates the length of a leaf blade of comparison variety ‘MCC02’. FIG. 13B illustrates the width of a leaf blade of comparison Variety ‘MCC02’. FIG. 13C illustrates the shape of base of a leaf blade of comparison variety ‘MCC02’. FIG. 13D illustrates the intensity of green color of a leaf blade of comparison variety ‘MCC02’. FIG. 13E illustrates the shape of apex of a leaf blade of comparison variety ‘MCC02’. FIG. 13F illustrates the colors of young leaves of comparison variety ‘MCC02’ at the stages soon, early, and medium.

FIGS. 14A-14G illustrate flowers of comparison variety ‘MCC02’. FIG. 14A illustrates the anthocyanin coloration of pedicel of a flower of comparison variety ‘MCC02’. FIG. 14B illustrates the anthocyanin coloration of pedicel of a flower of comparison variety ‘MCC02’. FIG. 14C illustrates the length of sepal of a flower of comparison variety ‘MCC02’. FIG. 14D illustrates the width of sepal of a flower of comparison variety ‘MCC02’. FIG. 14E illustrates the anthocyanin coloration of sepal of a flower of comparison variety ‘MCC02’. FIG. 14F illustrates the color of ligula of a flower of comparison variety ‘MCC02’. FIG. 14G illustrates the anthocyanin coloration of staminode of a flower of comparison variety ‘MCC02’.

FIGS. 15A-15J illustrate fruit of comparison variety ‘MCC02’. FIG. 15A illustrates the shape of a fruit of comparison variety ‘MCC02’. FIG. 15B illustrates the basal constriction of a fruit of comparison variety ‘MCC02’. FIG. 15C illustrates the shape of apex of a fruit of comparison variety ‘MCC02’. FIG. 15D illustrates the fruit length of comparison variety ‘MCC02’. FIG. 15E illustrates the fruit diameter of comparison variety ‘MCC02’. FIG. 15F illustrates the fruit surface of comparison variety ‘MCC02’. FIG. 15G illustrates the fruit depth of grooves of comparison variety ‘MCC02’. FIG. 15H illustrates the fruit color of comparison variety ‘MCC02’. FIG. 15I illustrates the exocarp thickness of a fruit of comparison variety ‘MCC02’. FIG. 15J illustrates the color of pulp of a fruit of comparison variety ‘MCC02’.

FIGS. 16A-16E illustrate seeds of comparison variety ‘MCC02’. FIG. 16A illustrates the shape in longitudinal section of a seed of comparison variety ‘MCC02’. FIG. 16B illustrates the seed length of comparison variety ‘MCC02’. FIG. 16C illustrates the seed width of comparison variety ‘MCC02’. FIG. 16D illustrates the seed thickness of comparison variety ‘MCC02’. FIG. 16E illustrates the seed cotyledon color of comparison variety ‘MCC02’.

FIG. 17 illustrates rates of survival for self-pollinated individuals during progress testing of variety ‘ISC-3’. “REP” represents replicate experiments 1-7 in the leftmost column. The number of flowers initially pollinated is shown in the column second from left. The number of surviving flowers for each replicate are displayed in columns from left to right, tracking the number of survivors at 3-, 5-, 8-, 15-, and 30-days post-pollination, respectively. The final rate of survival is shown in the rightmost column, calculated as the number of survivors at 30-days post-pollination divided by the number of flowers initially pollinated. The grand survival rate for each column is shown as a percentage across the bottom.

FIGS. 18A-18G illustrate the superior yield of the claimed self-compatible cacao clones across crop years. FIGS. 18A, C, and F illustrate cumulative yields for select self-compatible cacao clones across crop years, wherein each colored series of data points represents a different cacao lineage. Self-compatible lineages are denoted by labeled arrows, and comparative commercial variety ‘MCC02’ is illustrated in orange (FIG. 18A) or black (FIGS. 18C, and F). FIGS. 18B, D, E, and G illustrate yield per year for many individual clones for each of two (FIG. 18G) or three (FIGS. 18B, D, and E) crop years, wherein the measured clones are illustrated by orange bars, and the green bar illustrates the yield of comparative commercial variety ‘MCC02’. FIGS. 18A-18B illustrate yield for Clone Trial 95 (“CT95”), measuring 1,500 trees/Ha planted in May 2018. FIGS. 18C-18D further illustrate CT95, measuring 2,000 trees/Ha. Variety ‘ISC-2’ is represented in FIGS. 18A-18D by the clone labeled “44” (indicated with asterisks). Variety ‘ISC-3’ is represented in FIGS. 18A-18D by the clone labeled “5” (indicated with x marks). FIG. 18E illustrates yield for Clone Trial 107 (“CT107 ”) planted in August 2018 FIGS. 18F-18G illustrate yield for Clone Trial 162 (“CT162”) planted in May 2019. Variety ‘ISC-1’ is represented in FIG. 18G by the clones labeled “2” (circled in red).

FIGS. 19A-19C illustrate additional data demonstrating the superior yield of the claimed self-compatible cacao clones across crop years. FIG. 19A further illustrates cumulative yield (in Ton/Ha) from CT95 at 1,500 trees/Ha over crop age 2-6 years, with variety ‘ISC-2’ represented by the clone labeled “44|GTB11C18R33|MCC02×S1” (light purple), variety ‘ISC-3’ represented by the clone labeled “5|GTB02C38R21|MCC02×RUQ1347” (green), and reference variety ‘MCC02’ represented by the clone labeled “MCC02” (dark purple). FIG. 19B further illustrates cumulative yield (in Ton/Ha) from CT162 over crop age 2-5 years, with variety ‘ISC-1’ represented by the clone labeled “2|GTB04C09R19|MCC02×RUQ 1347” (green), and reference variety ‘MCC02’ represented by the clone labeled “MCC02” (dark purple). FIG. 19C further illustrates cumulative yield (in Ton/Ha) from CT95 at 2,000 trees/Ha over crop age 2-6 years, with variety ‘ISC-2’ represented by the clone labeled “44|GTB11C18R33|MCC02×S1” (light purple), variety ‘ISC-3’ represented by the clone labeled “5|GTB02C38R21|MCC02×RUQ1347” (green), and reference variety ‘MCC02’ represented by the clone labeled “MCC02” (dark purple).

FIGS. 20A-20B illustrate rates of survival for self-pollinated individuals during progress testing of varieties ‘ISC-1’ (FIG. 20A) and ‘ISC-2’ (FIG. 20B). “REP” represents replicate experiments 1-6 in the leftmost column. The number of flowers initially pollinated is shown in the column second from left. The number of surviving flowers for each replicate are displayed in columns from left to right, tracking the number of survivors at 3-, 5-, 8-, 15-, and 30-days post-pollination, respectively. The final rate of survival is shown in the second-from-right column, calculated as the number of survivors at 30-days post-pollination divided by the number of flowers initially pollinated. The grand survival rate for each column is shown as a percentage across the bottom. The average survival rates are shown in the rightmost column. The standard deviation (StDev) is shown in the bottom right corner. FIG. 20A illustrates the progress testing survival rate data for variety ‘ISC-1’. FIG. 20B illustrates the progress testing survival rate data for variety ‘ISC-2’.

### DETAILED BOTANICAL DESCRIPTION

The following description is based on observations taken of plants and fruits of the new and distinctive cacao varieties of the present invention.

The dry weight of a single bean is calculated by dividing the dry bean weight by the number of healthy beans harvested. The yield is calculated by dividing the total fresh bean weight by the number of reps and multiplying by the plant density (1,175 trees/ha, unless indicated otherwise).

### Variety ‘ISC-1’

In certain aspects, the present invention relates to a new and distinctive cacao plant designated as ‘ISC-1’. Cacao variety ‘ISC-l’ has shown uniformity and stability for the traits, within the limits of environmental influence for the traits. Cacao ‘ISC-1’ has been increased with continued observation for uniformity. No variant traits have been observed or are expected in ‘ISC-1’.

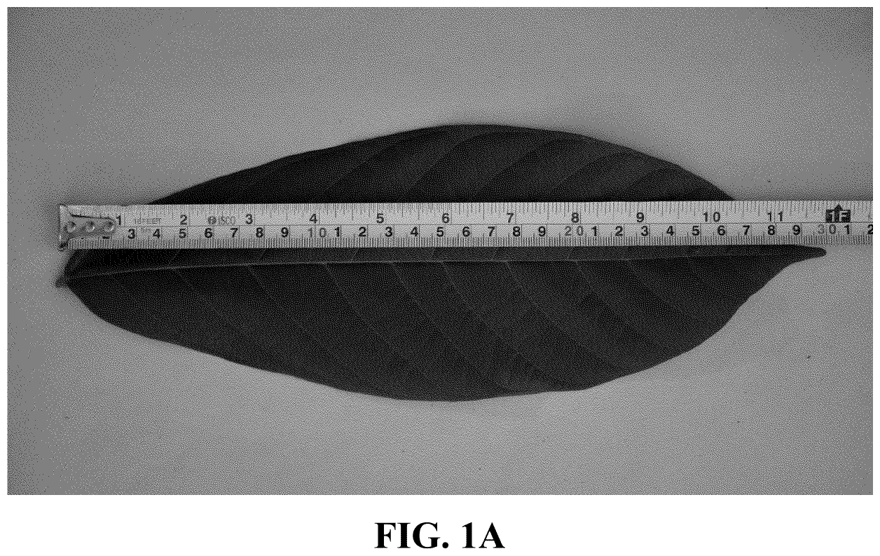

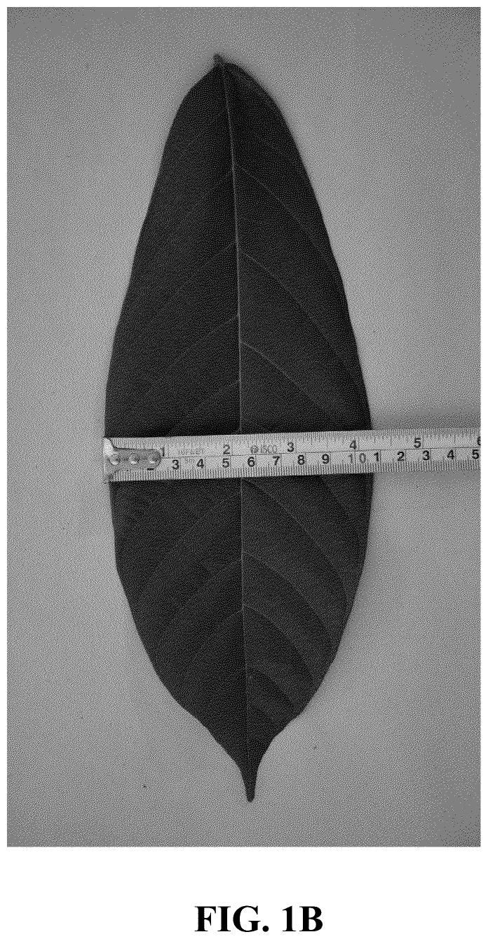

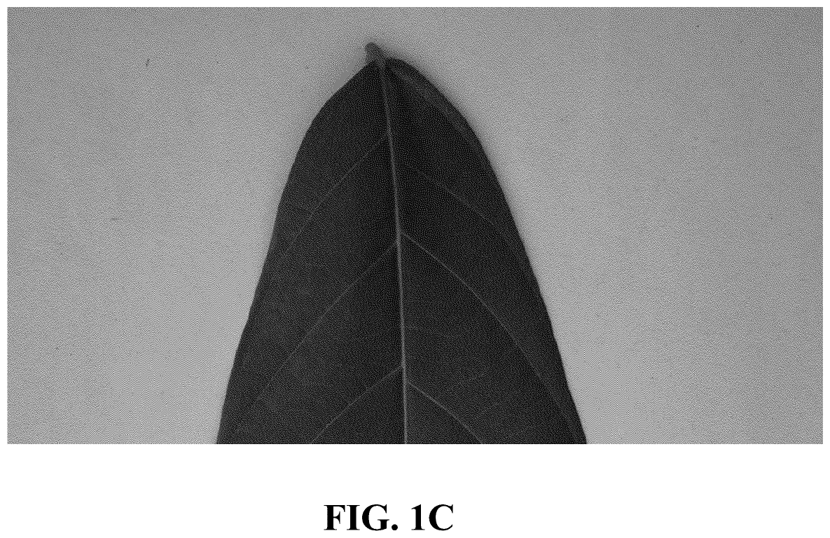

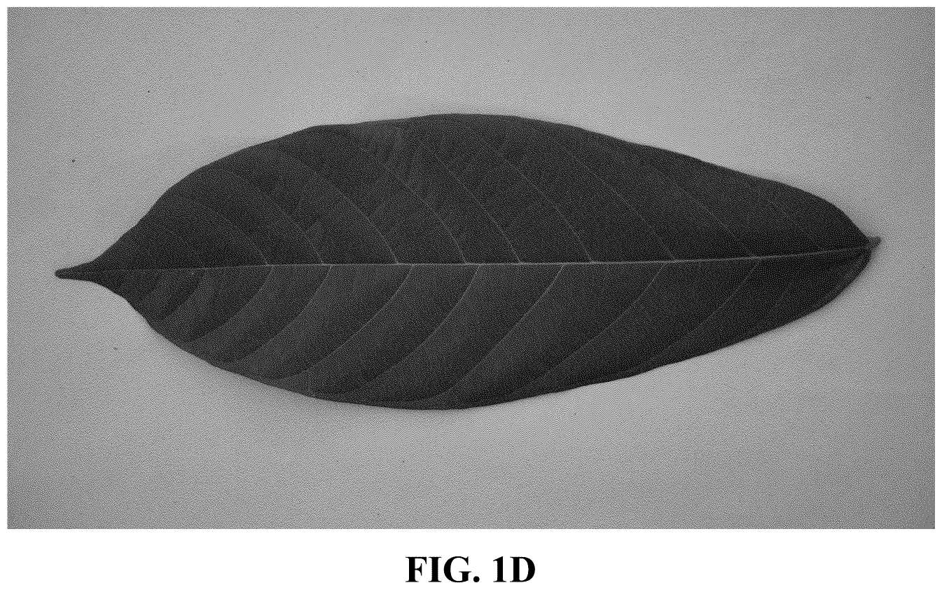

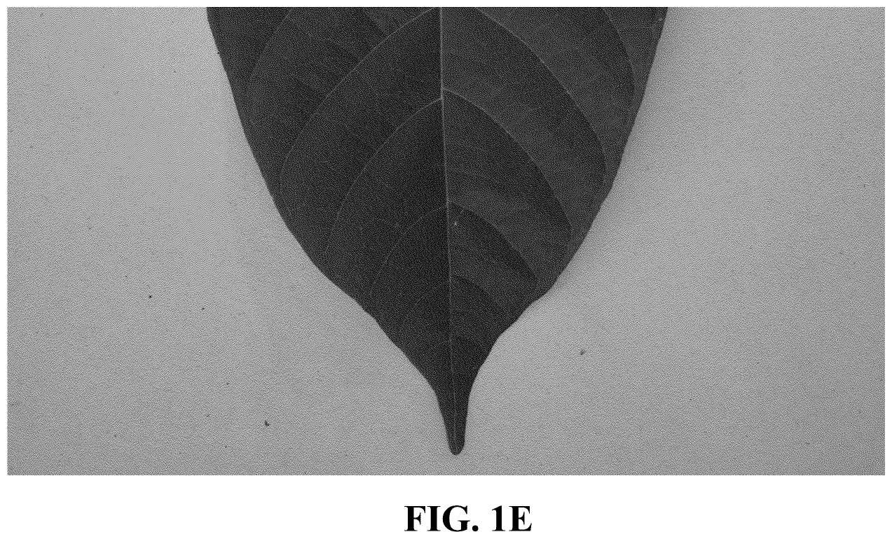

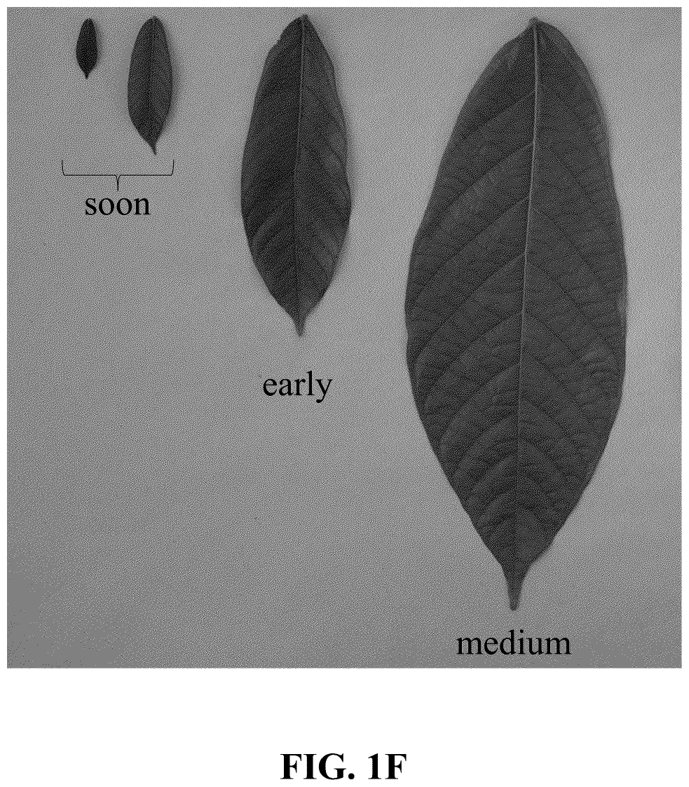

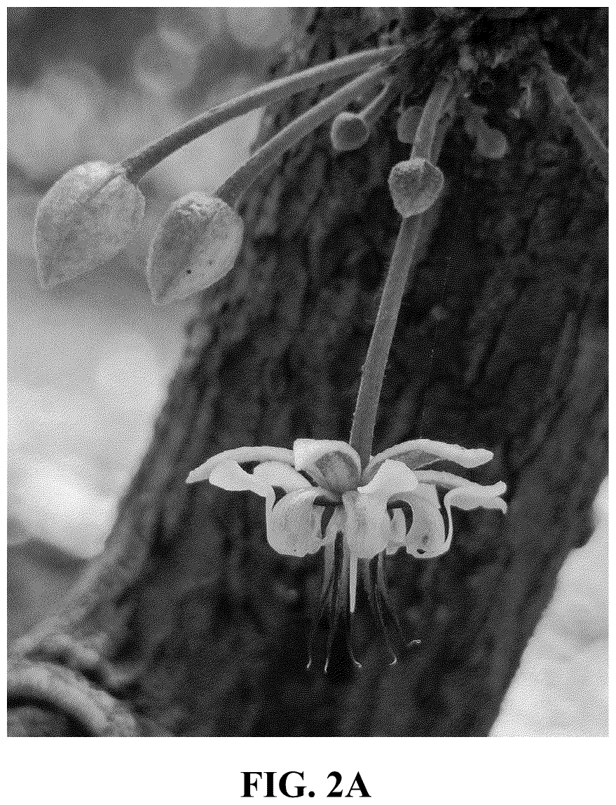

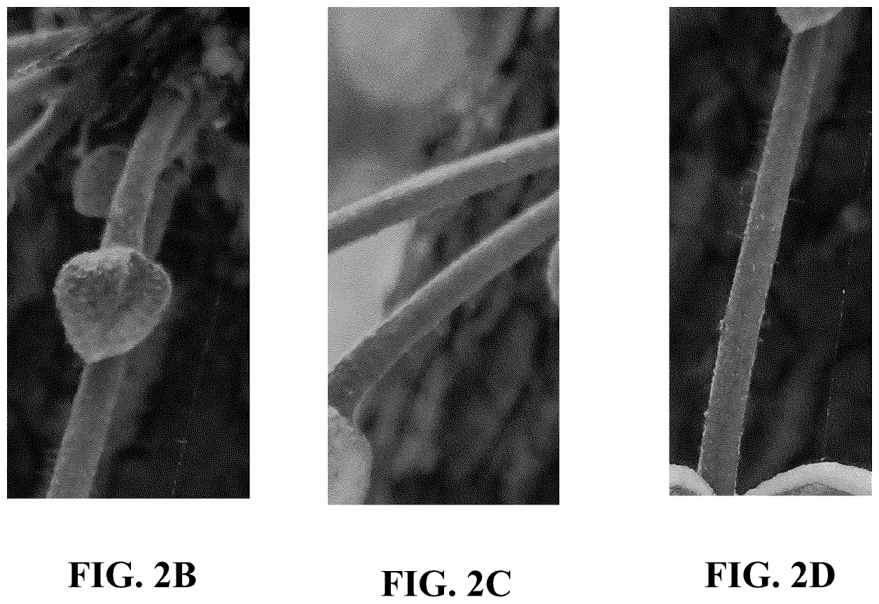

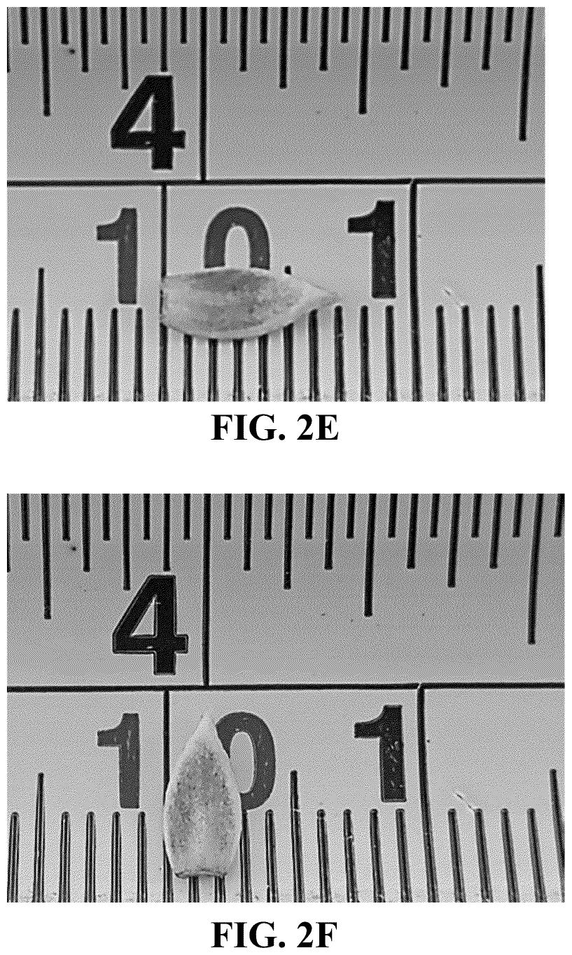

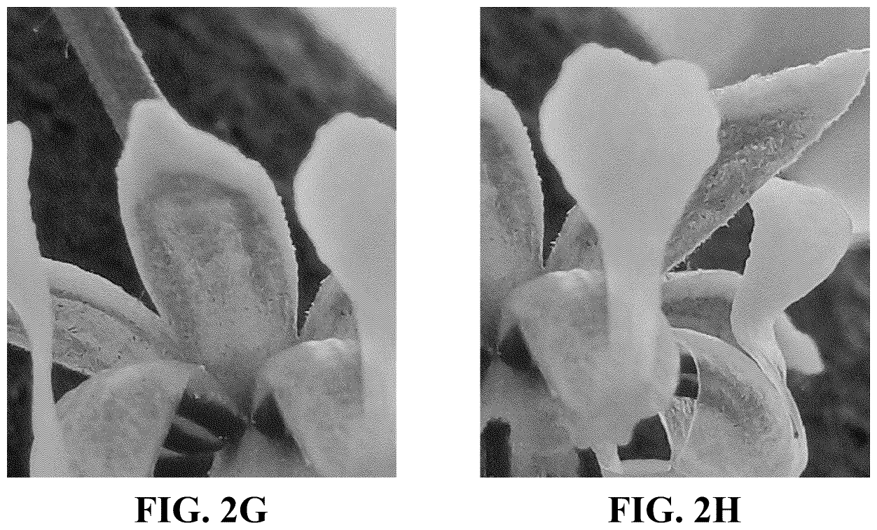

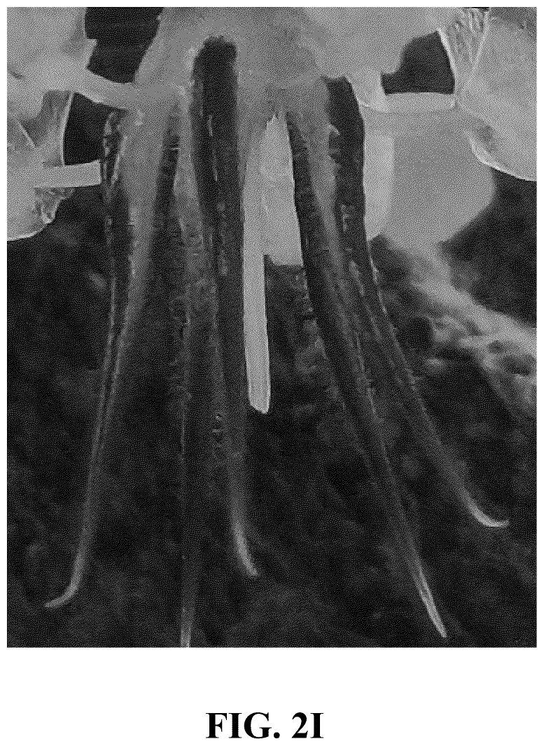

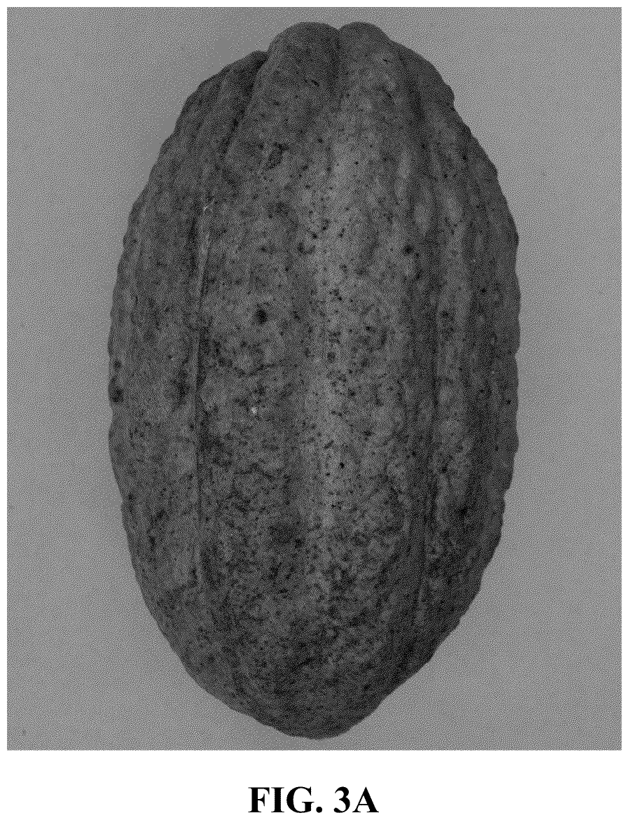

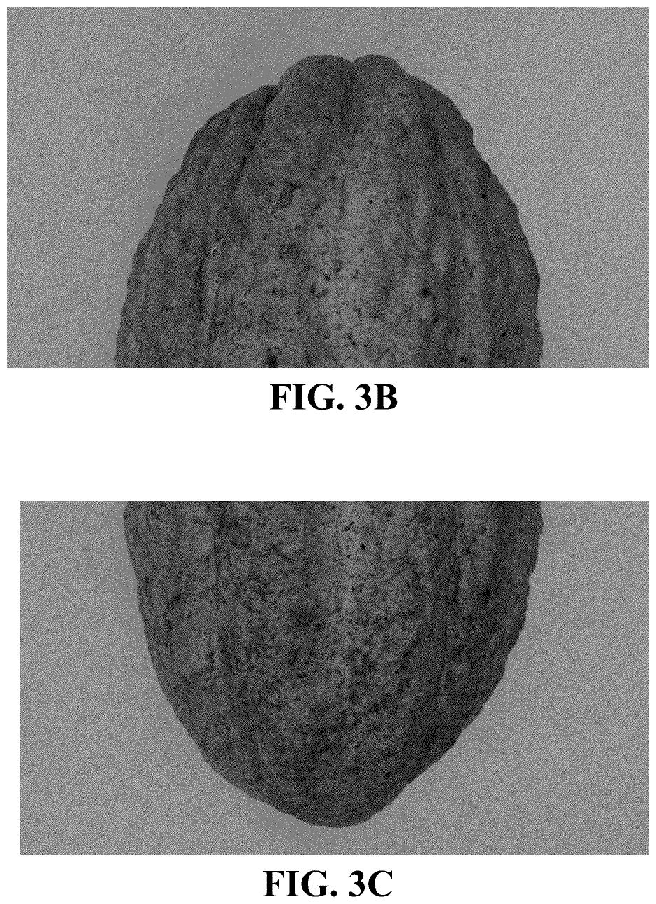

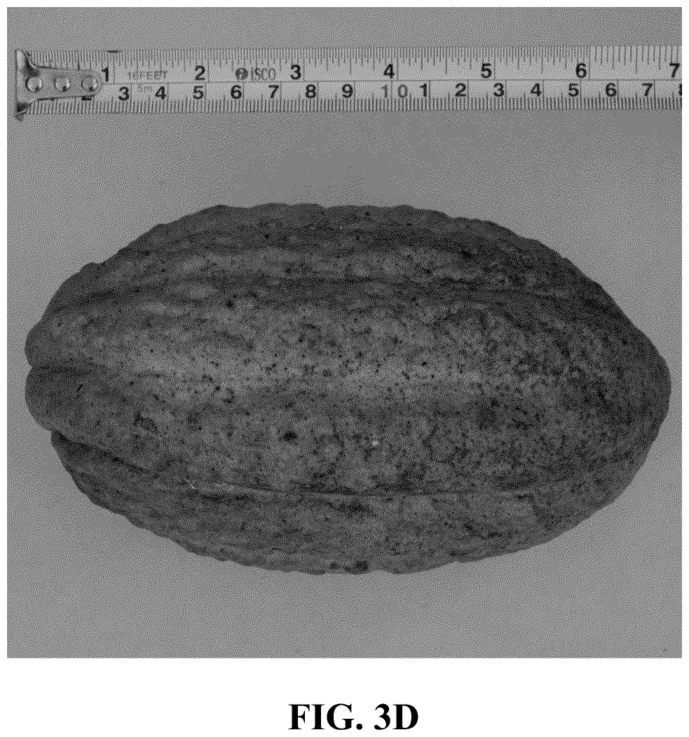

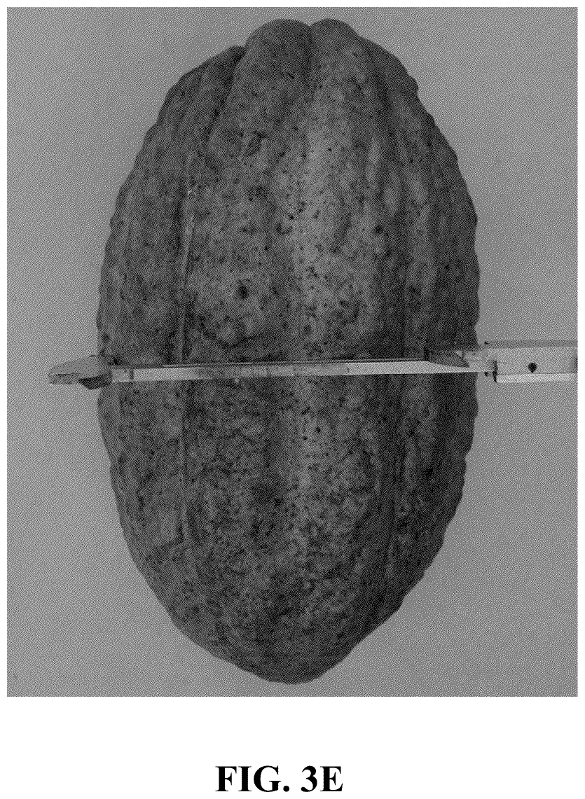

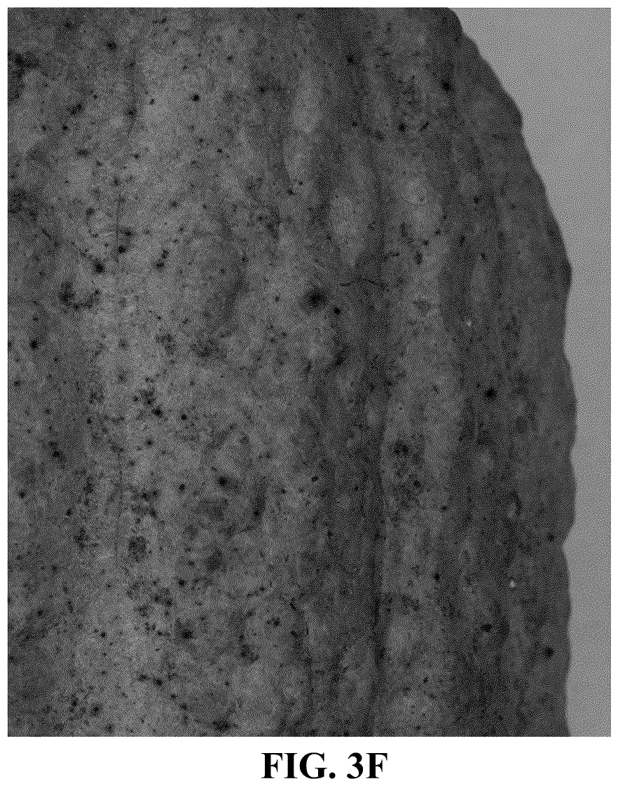

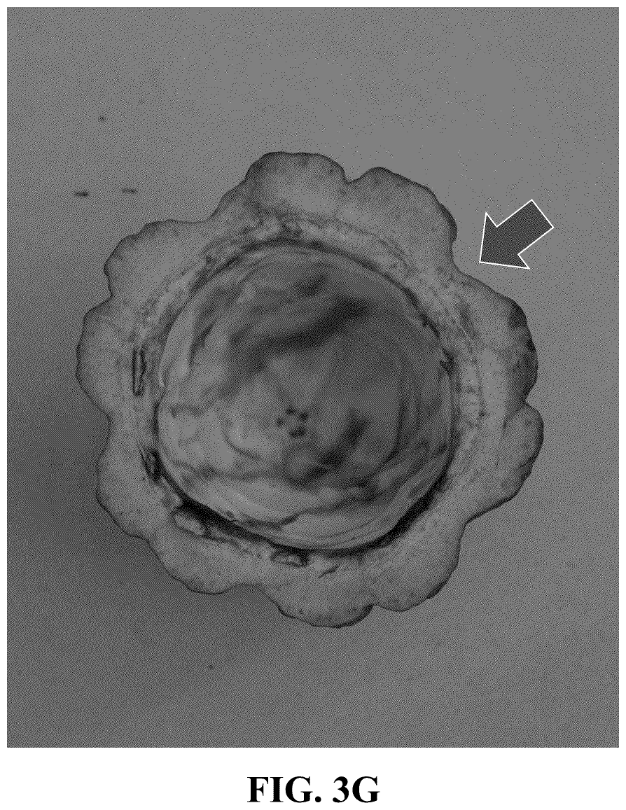

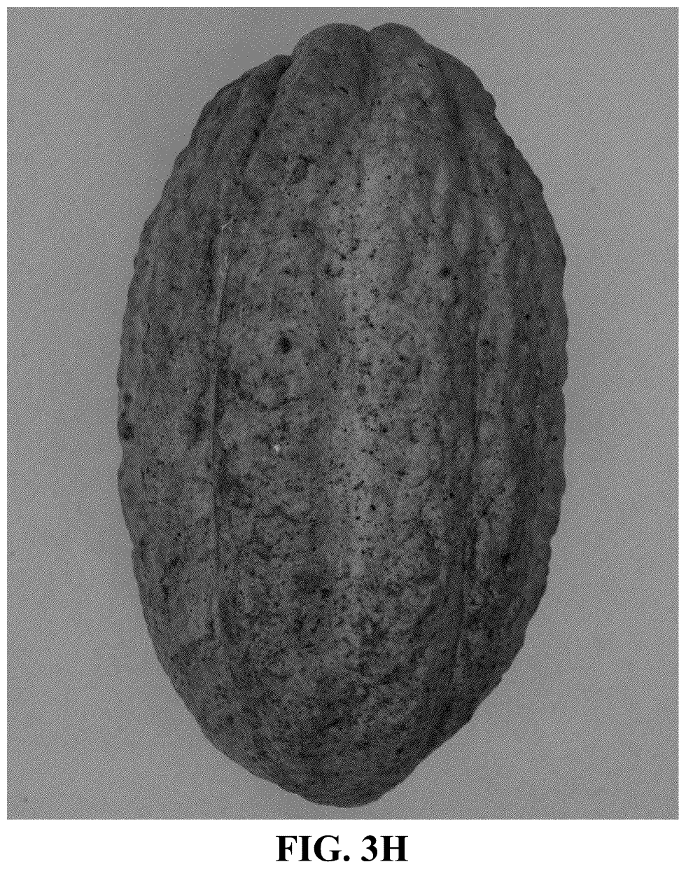

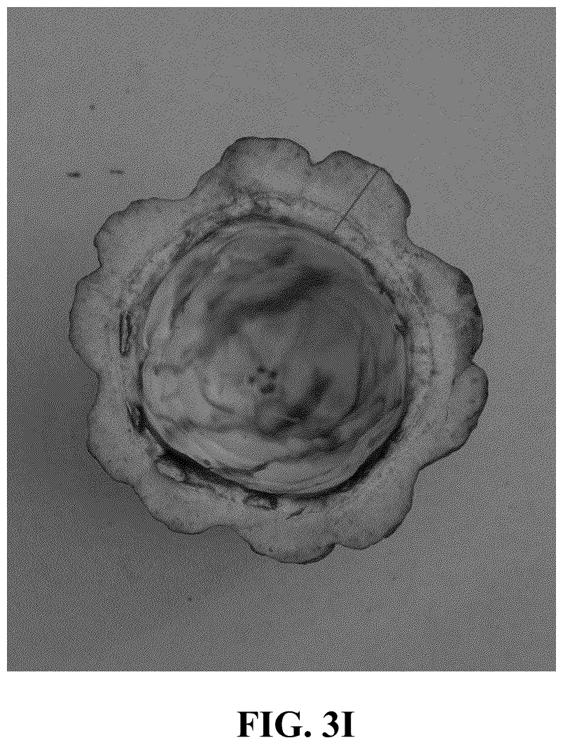

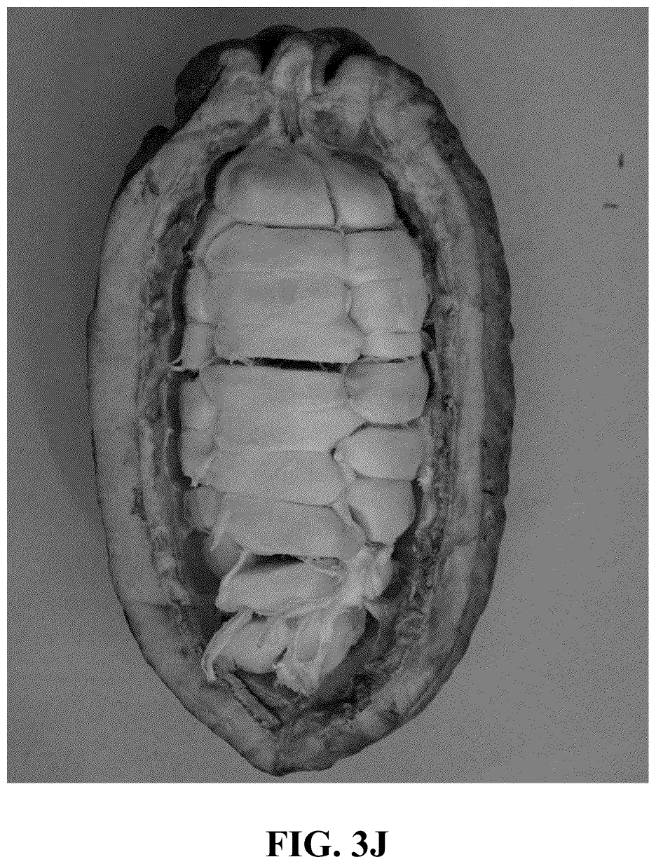

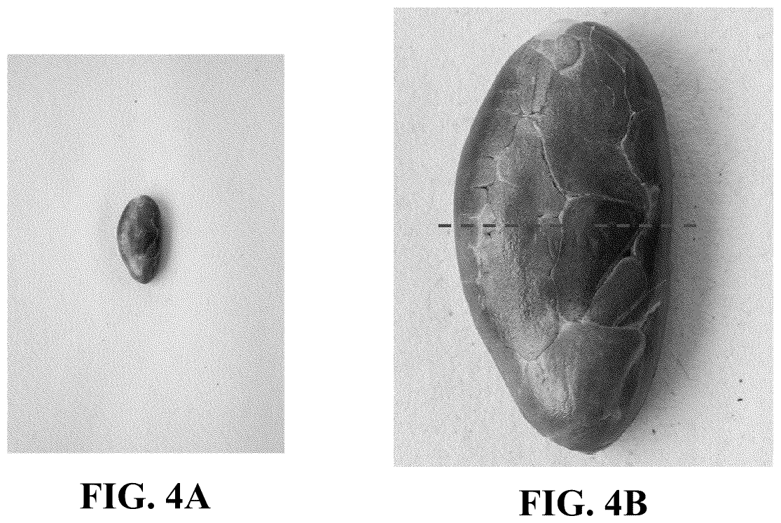

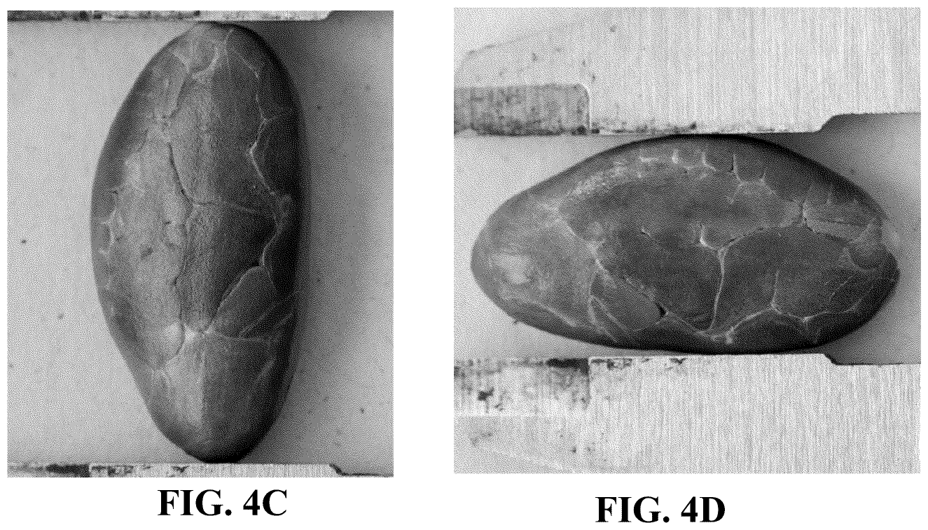

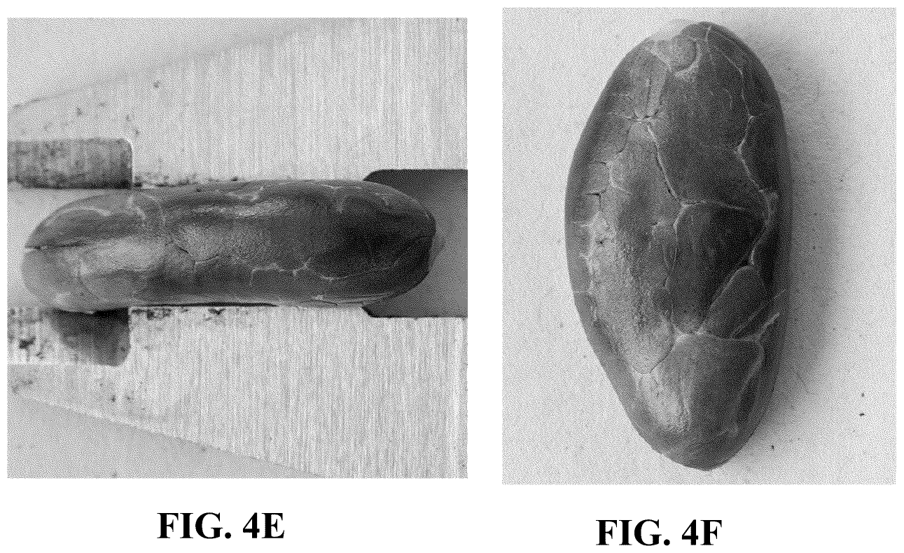

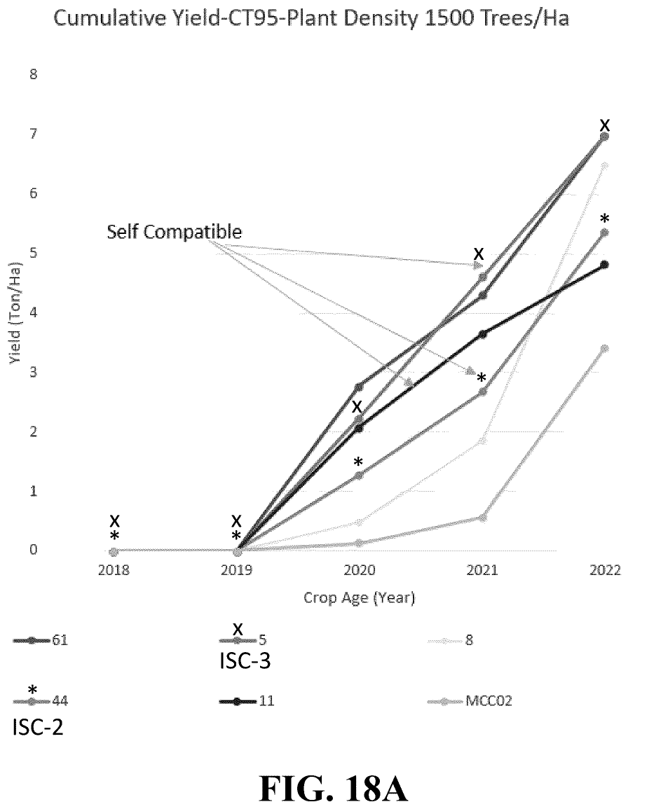

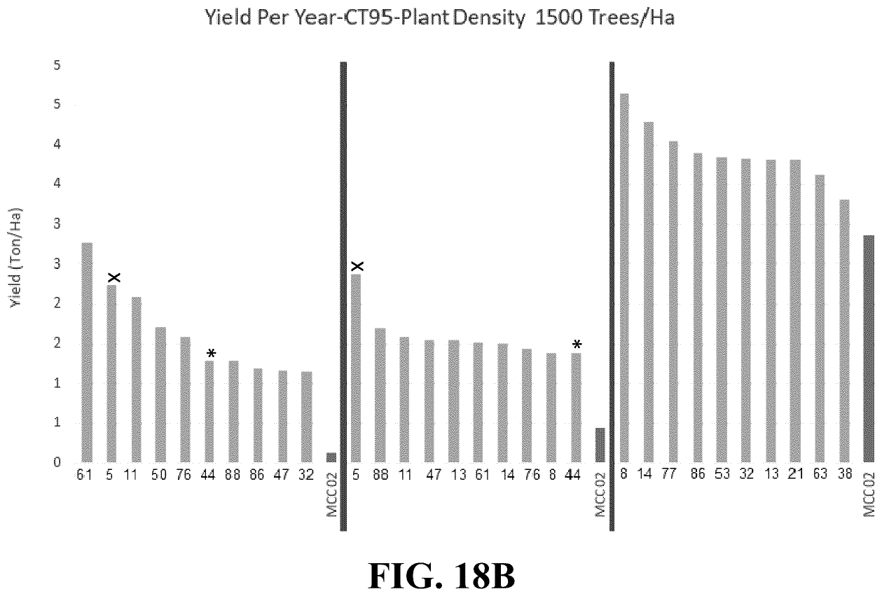

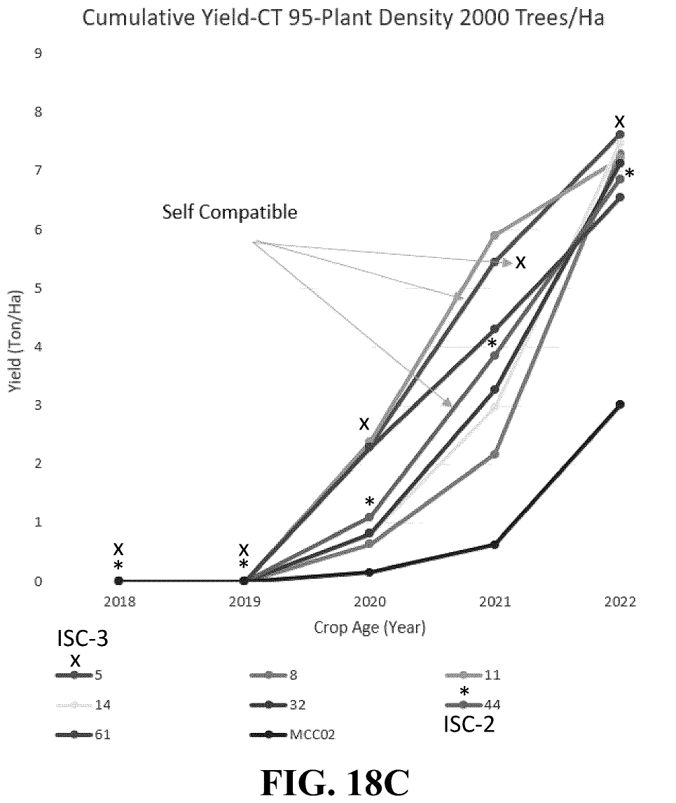

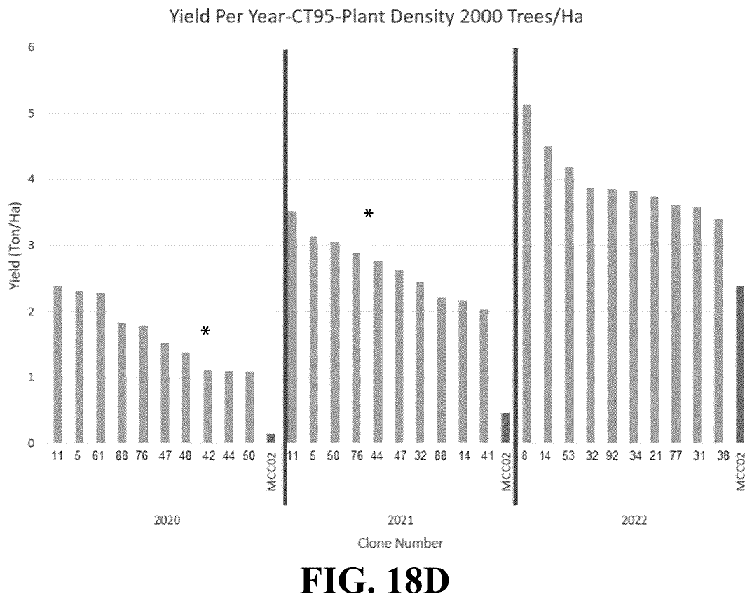

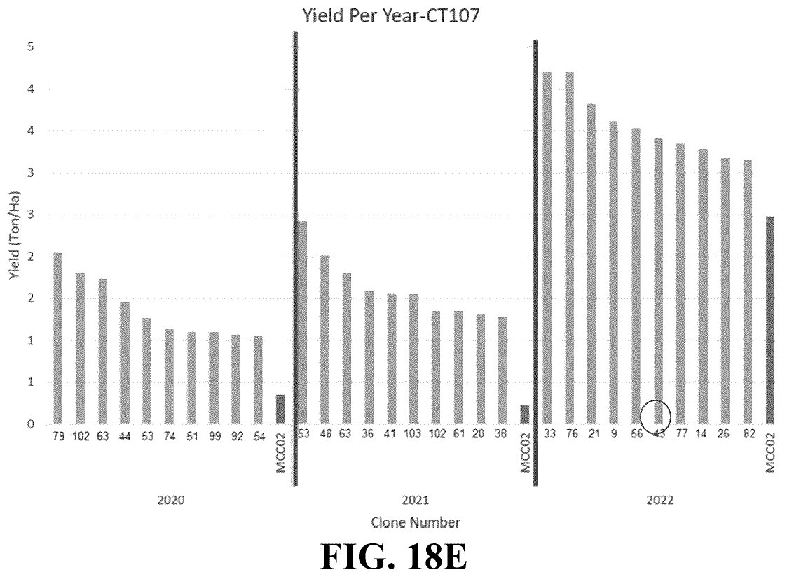

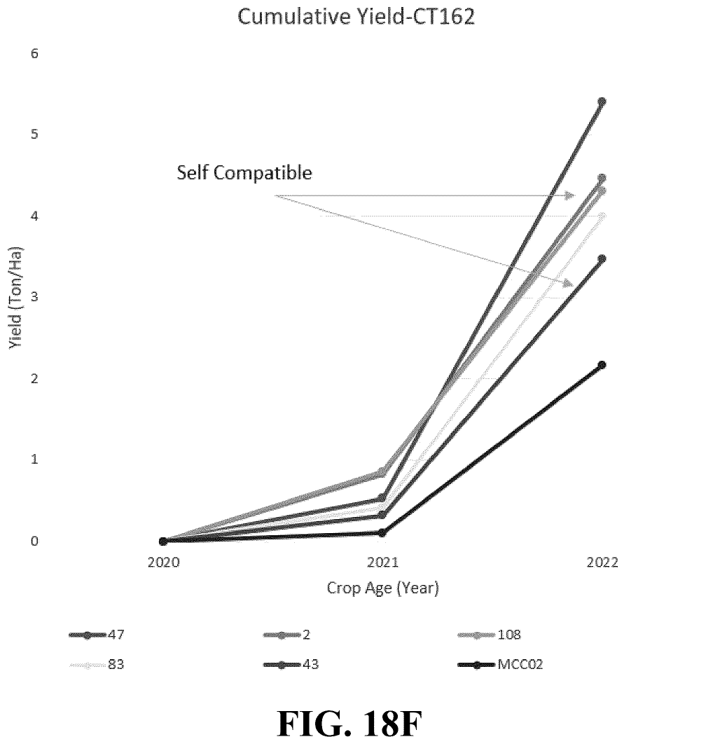

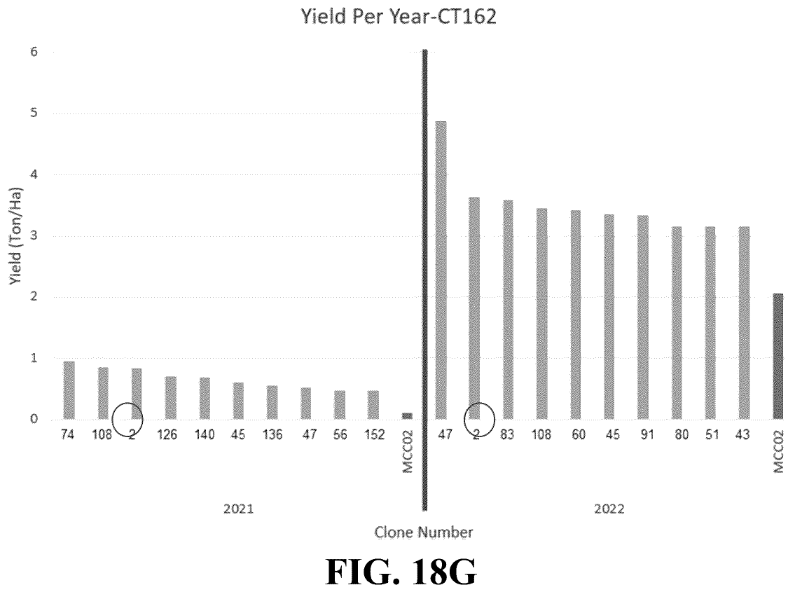

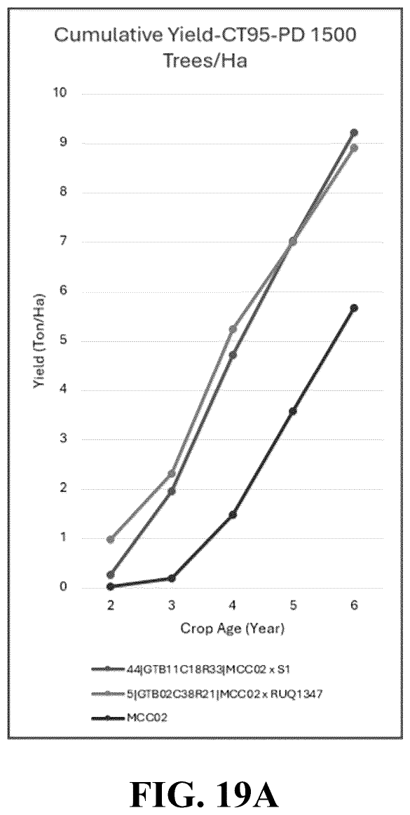

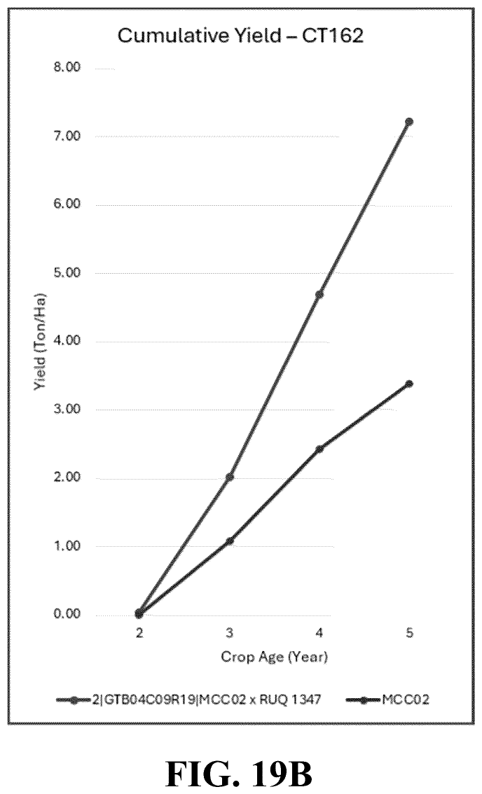

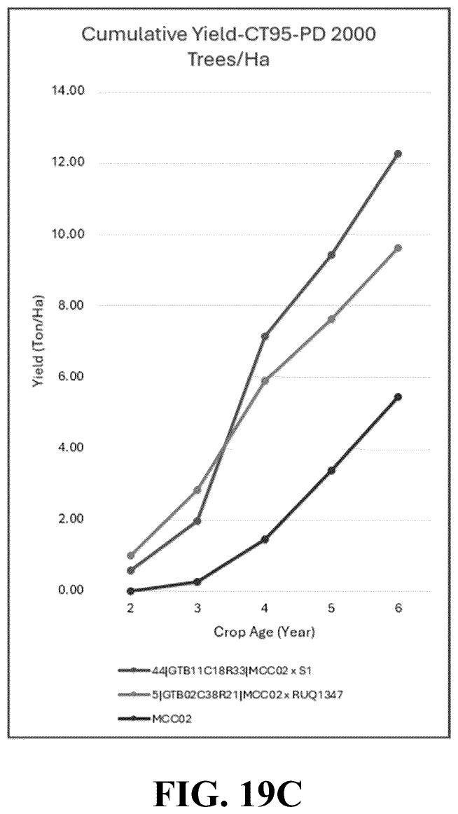

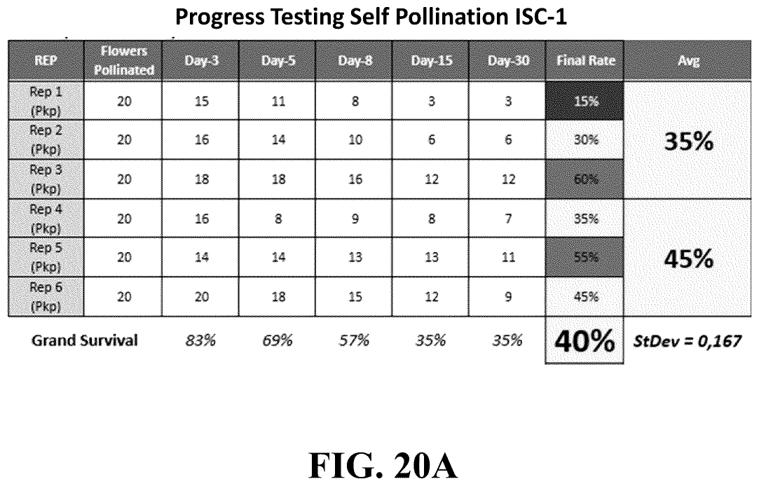

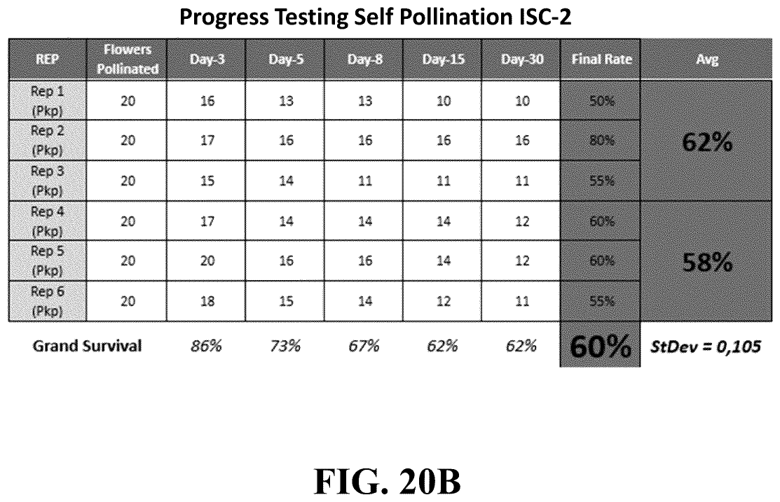

The variety description information in Table 1 below provides a summary of variety ‘ISC-1’ plant characteristics. Those of skill in the art will recognize that these are typical values that may vary due to environment, and that other values that are substantially equivalent are within the scope of the invention. The terminology and descriptors are in line with the descriptors of the “UPOV Guidelines for the Conduct of Tests for Distinctness, Uniformity, and Stability”, or the “Test Guidelines” for Theobroma cacao L. The “Test Guidelines” indicate reference varieties for the descriptors or characteristics that are included in the list. The terminology and descriptors used in these tables are in line with the official terminology as of the filing date, and are thus clear for a person skilled in the art. FIGS. 1A-4F illustrate the plant characteristics of variety ‘ISC-1’. FIGS. 18A-18G illustrate the success of the methods used to produce self-compatible variety ‘ISC-1’, and FIG. 18G illustrates the yield of variety ‘ISC-1’ as clone “2”. FIG. 19B illustrates additional data demonstrating the superior yield of variety ‘ISC-1’ across crop years (shown in green and labeled “2|GTB04C09R19|MCC02×RUQ 1347”). FIG. 20A illustrates the progress testing survival rate data for variety ‘ISC-1’.

The following detailed descriptions set forth the distinctive characteristics of ‘ISC-1’. The data which define these characteristics is based on observations taken in Pangkep, Indonesia. This description is in accordance with UPOV terminology. Color designations, color descriptions, and other phenotypical descriptions may deviate from the stated values and descriptions depending upon variation in environmental, seasonal, climatic, and cultural conditions. ‘ISC-1’ has not been observed under all possible environmental conditions. The indicated values represent averages calculated from measurements of several plants. Color references are primarily to The R.H.S. Colour Chart of The Royal Horticultural Society of London (R.H.S.) (2015 edition). Descriptive terminology follows the Plant Identification Terminology, An Illustrated Glossary, 2^{nd} edition by James G. Harris and Melinda Woolf Harris, unless where otherwise defined.

The cacao variety ‘ISC-1’ originated from a controlled cross between varieties ‘MCC02’and ‘RUQ1347’. The breeder's reference name of variety ‘ISC-1’ is ‘GTB04C09R19’. Cacao variety ‘ISC-1’ has subsequently been asexually propagated via grafting onto rootstock.
Classification:
   Species.—Theobroma cacao L.
   Common name.—Cacao.
   Denomination.—‘ISC-1’.
Parentage:
   Female parent.—‘MCC02’ (unpatented).
   Male parent.—‘RUQ1347’ (unpatented).

TABLE 1
‘ISC-1’.

| Characteristic | Value |
|---|---|
| Leaf blade: length | 30 cm |
| Leaf blade: width | 10.7 cm |
| Leaf blade: shape of base | Obtuse |
| Leaf blade: intensity of green color | Dark |
| Leaf blade: apex | Acuminate |
| Young leaf: color - soon | Brown-Dark red |
| Young leaf: color - early | Dark red |
| Young leaf: color - medium | Medium brown |
| Flower: anthocyanin coloration of pedicel | Strong-moderate |
| Flower: length of sepal | 0.7 cm |
| Flower: width of sepal | 0.3 cm |
| Flower: anthocyanin coloration of sepal | Moderate |
| Flower: color of ligula | Yellow |
| Staminode: anthocyanin coloration | Strong |
| Fruit: shape | Oblong |
| Fruit: basal constriction | Absent/Very Weak |
| Fruit: shape of apex | Obtuse |
| Fruit: length | 17.07 cm |
| Fruit: diameter | 10.31 cm |
| Fruit: length/diameter ratio | 1.66:1 |
| Fruit: surface | Moderately rough |
| Fruit: depth of groove | Deep |
| Fruit: color | Medium red |
| Fruit: exocarp thickness | Thick; 2.08 cm |
| Fruit: color of pulp | White |
| Fruit: sweetness of pulp | μ19.72° Brix |
| Fruit: number of seeds | Pod 1: 39 seeds |
| | Pod 2: 35 seeds |
| Seed: shape in longitudinal section | Elliptic |
| Seed: length | 3.04 cm |
| Seed: width | 1.54 cm |
| Seed: ratio length/width | 1.97:1 |
| Seed: thickness | 0.89 cm |
| Seed: cotyledon color | Purple |

The self-compatible phenotype of cacao variety ‘ISC-1’ has been confirmed through field observation by performing controlled (hand) self-pollinations.

### Variety ‘ISC-2’

In certain aspects, the present invention relates to a new and distinctive cacao plant designated as ‘ISC-2’. Cacao variety ‘ISC-2’ has shown uniformity and stability for the traits, within the limits of environmental influence for the traits. Cacao ‘ISC-2’ has been increased with continued observation for uniformity. No variant traits have been observed or are expected in ‘ISC-2’.

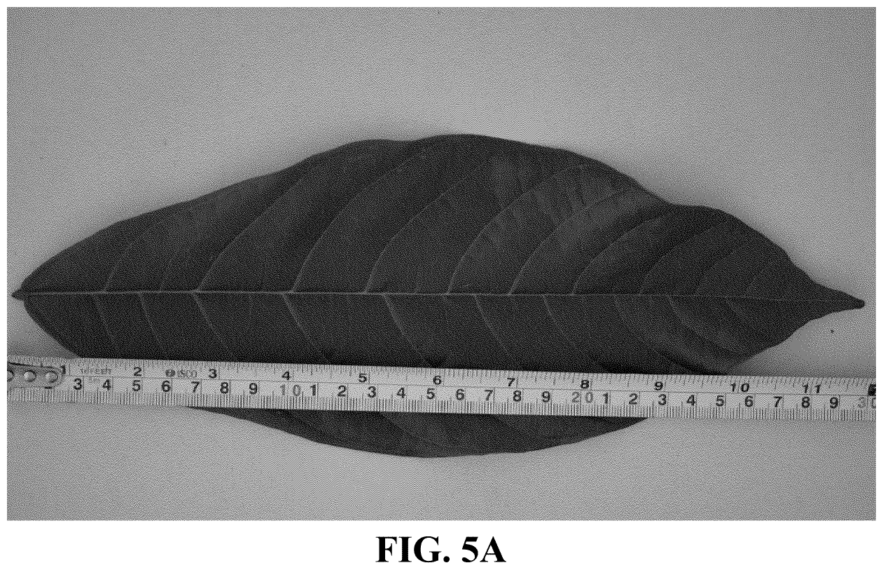

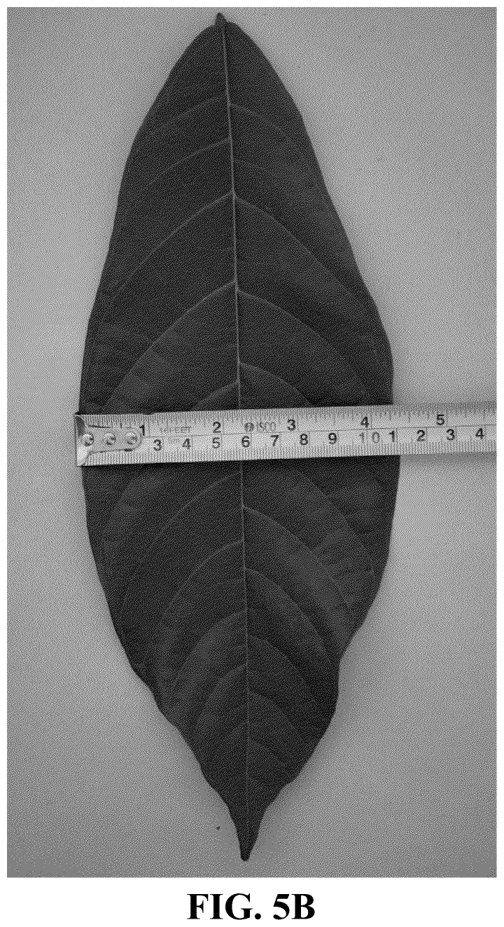

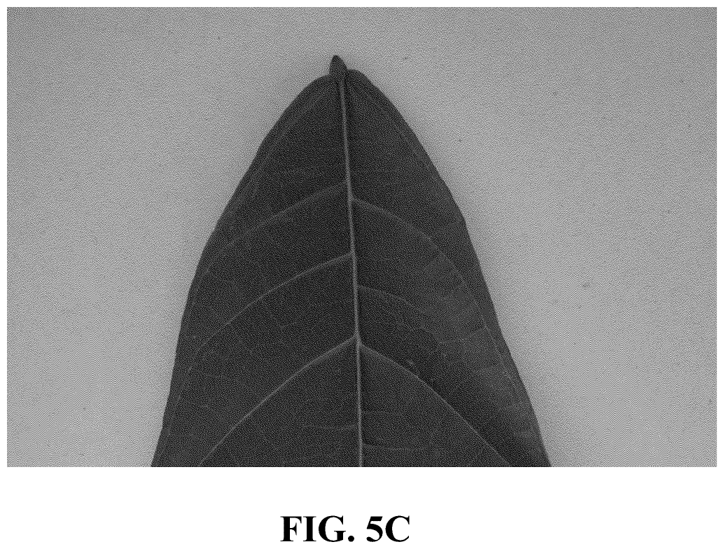

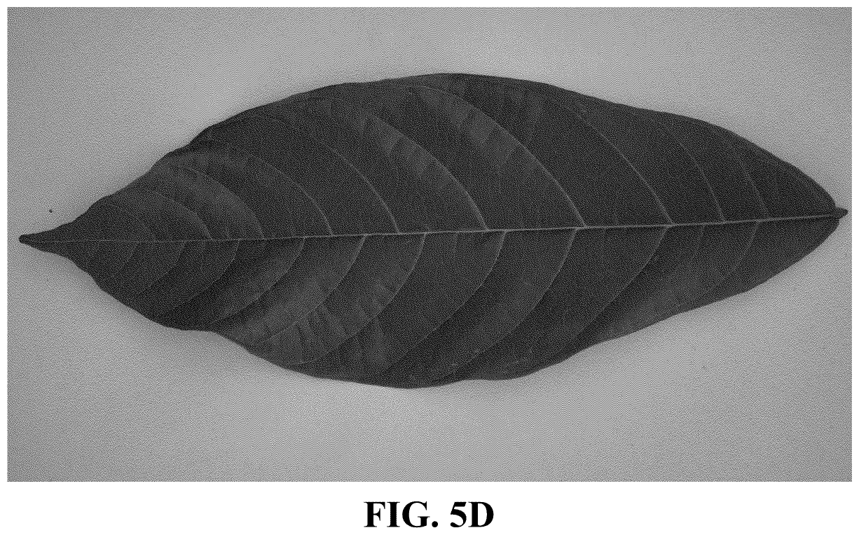

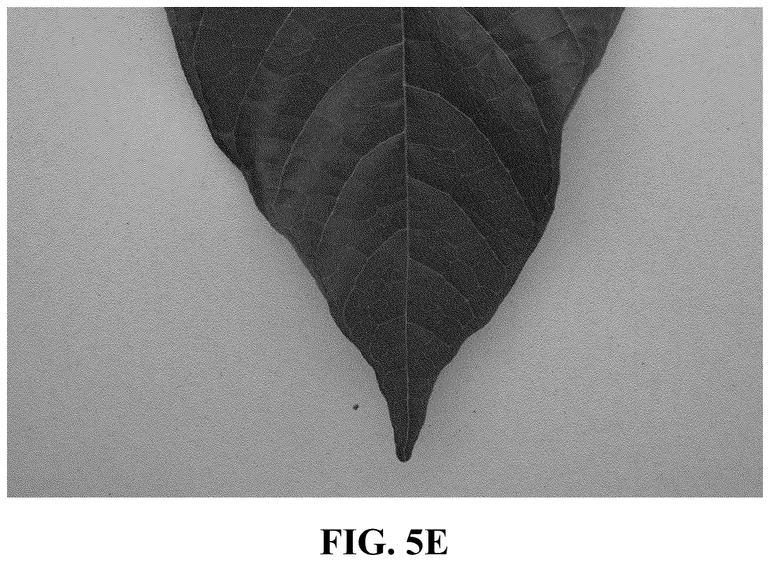

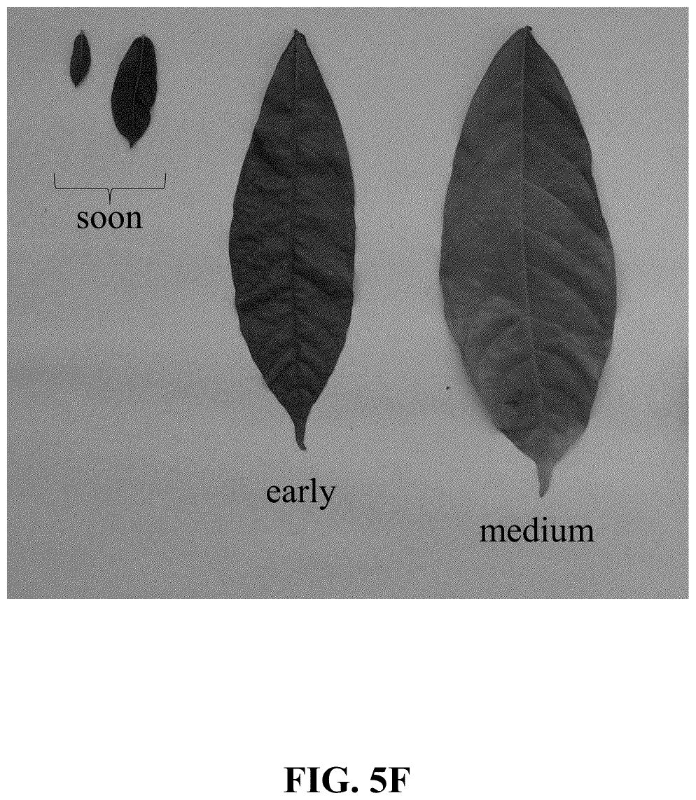

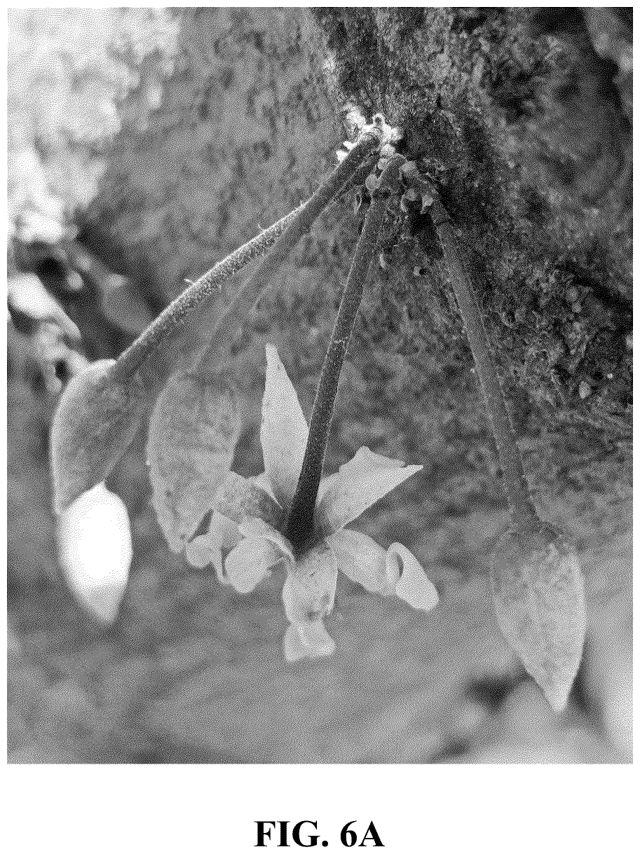

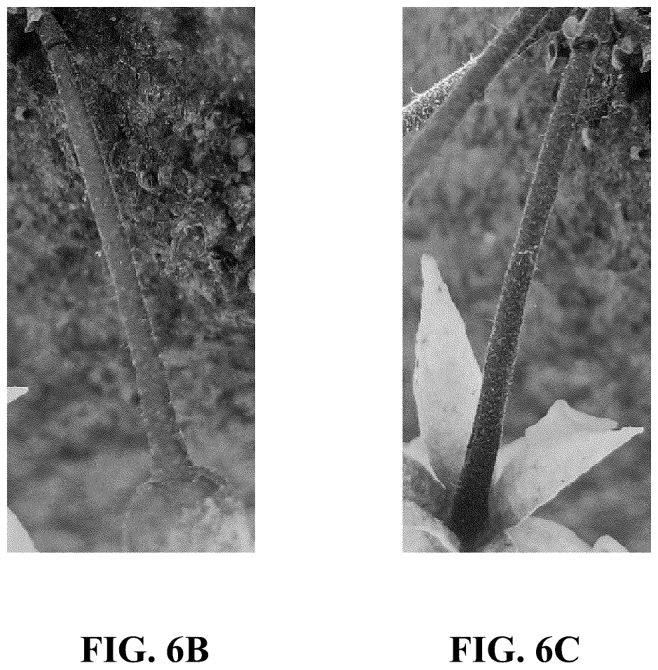

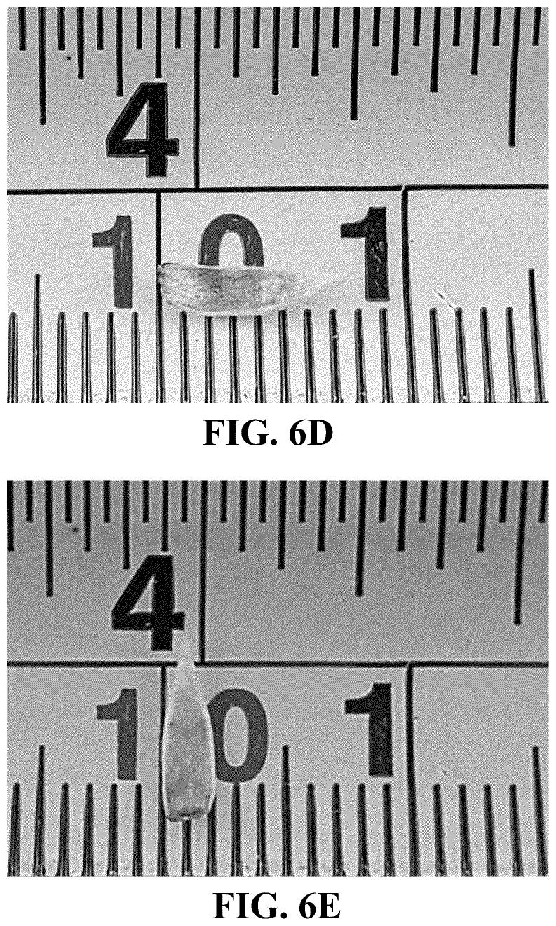

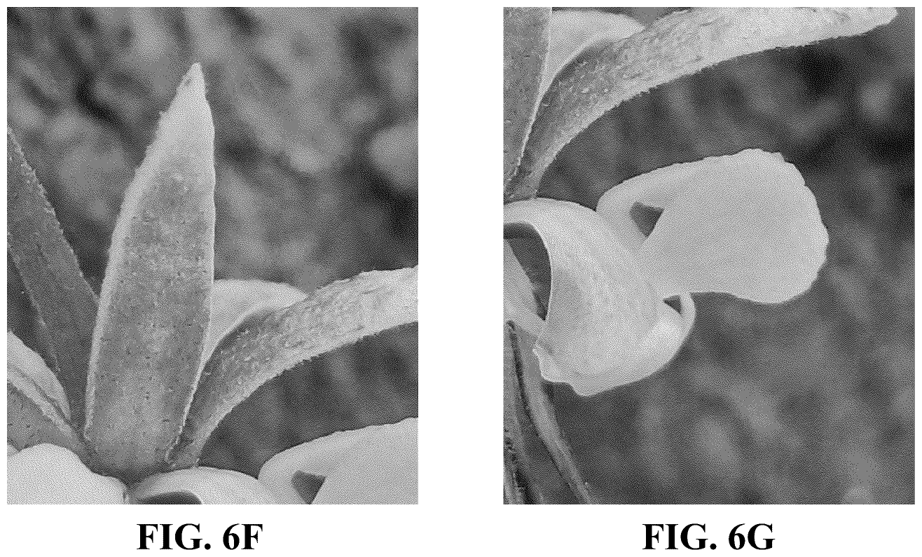

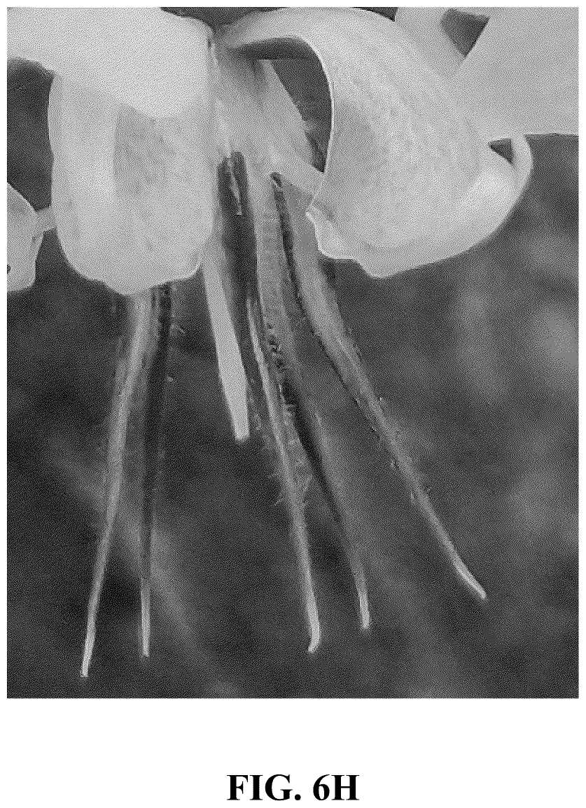

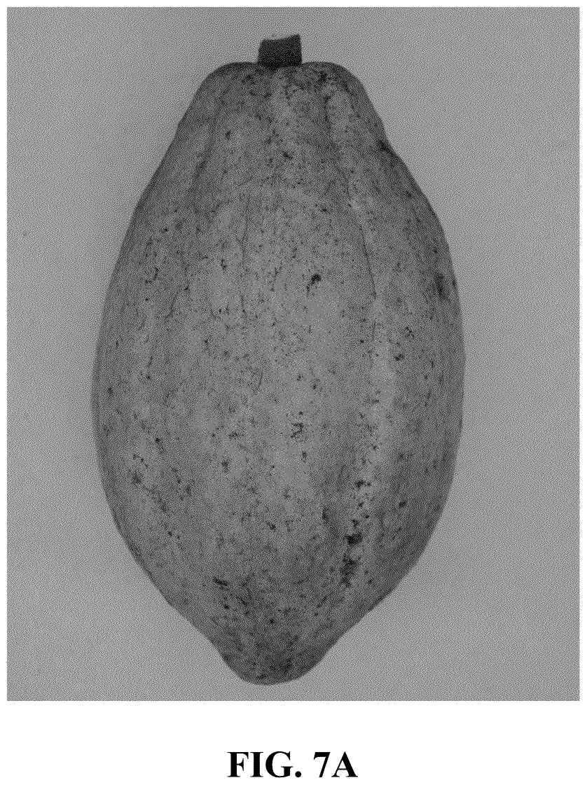

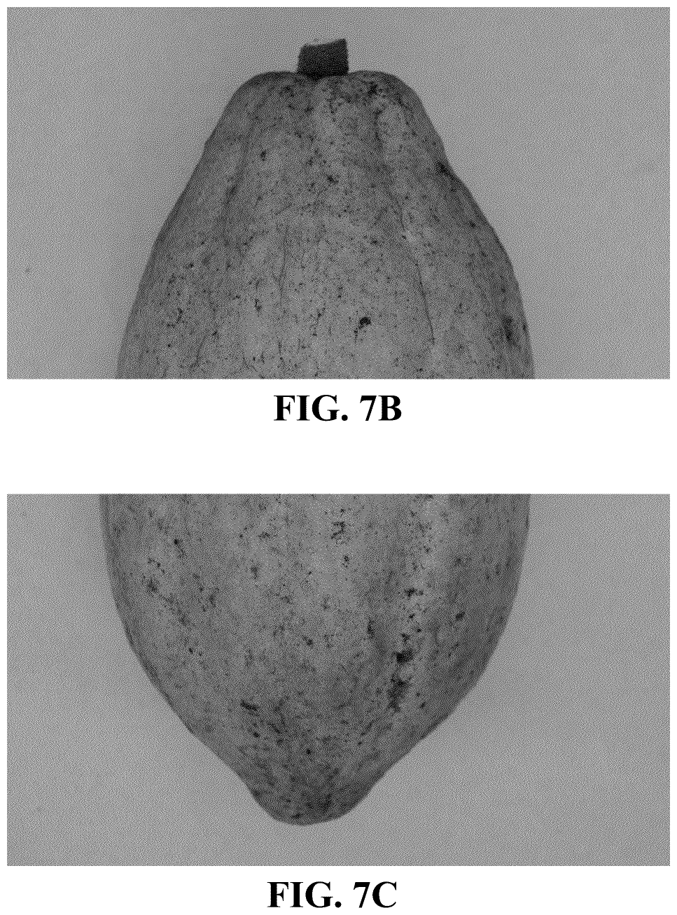

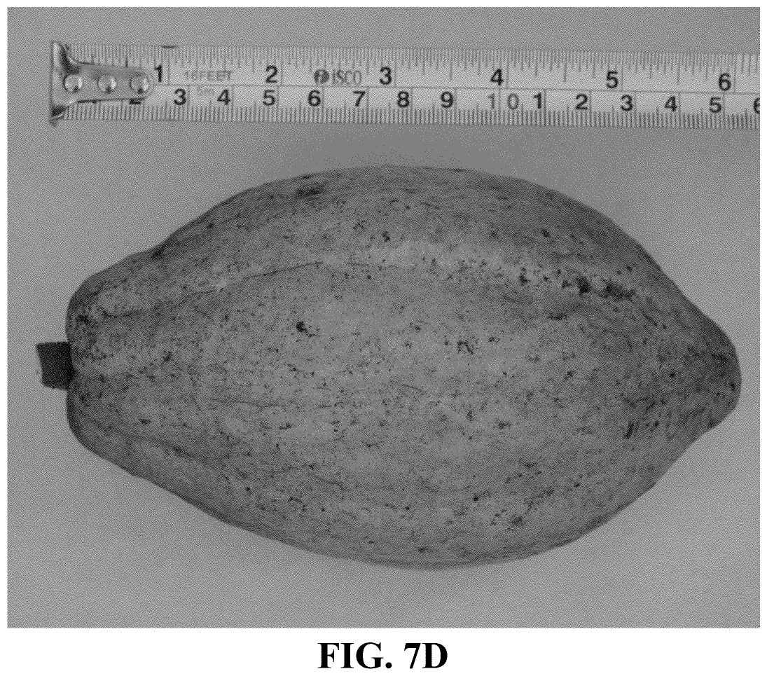

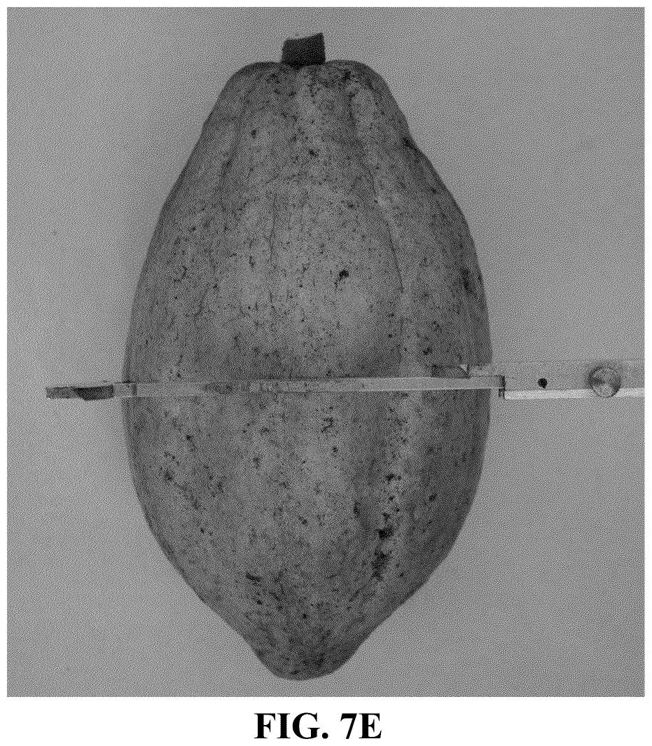

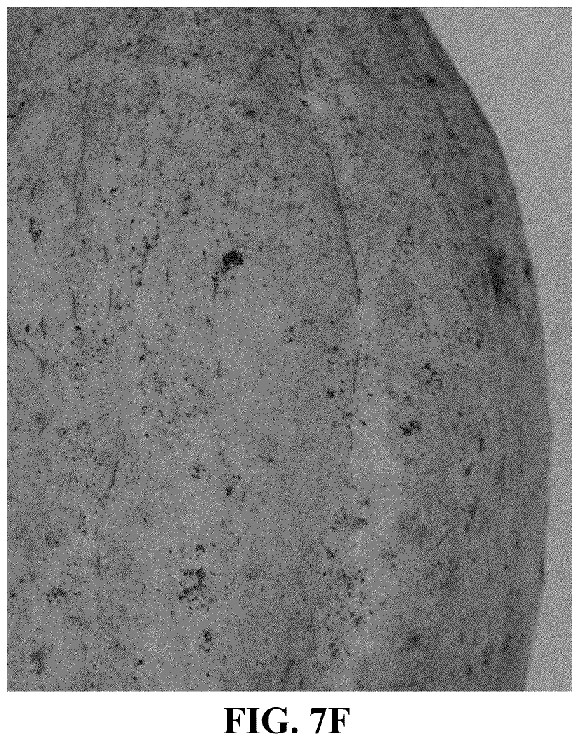

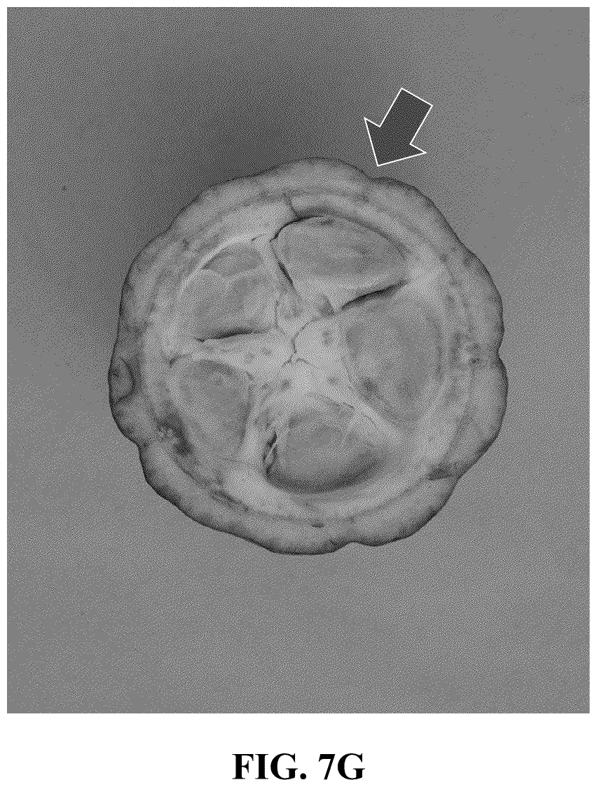

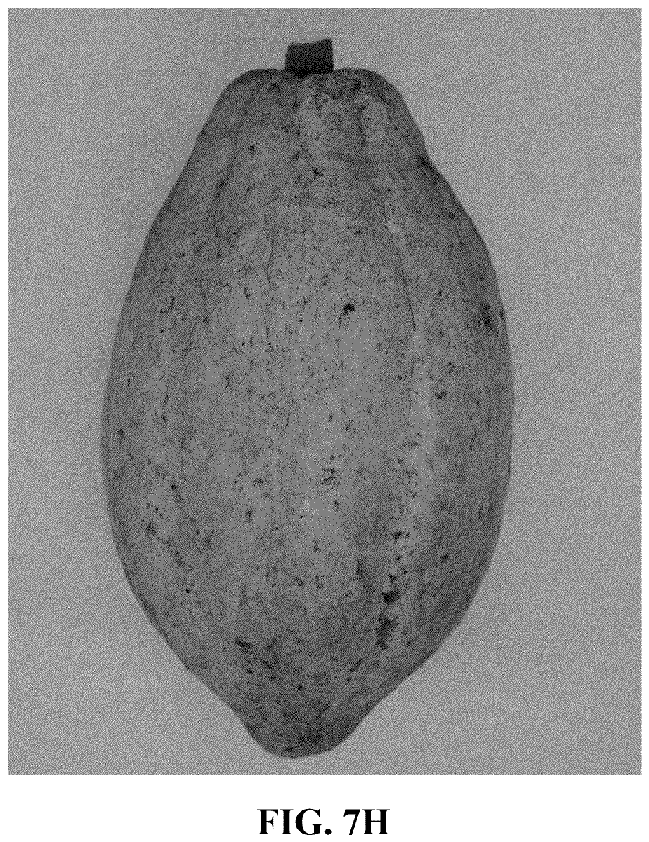

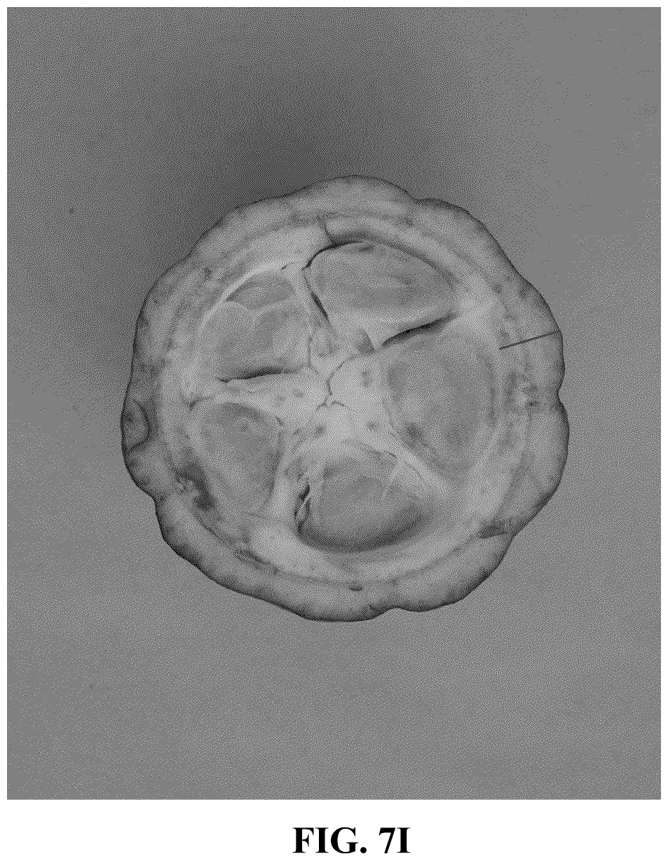

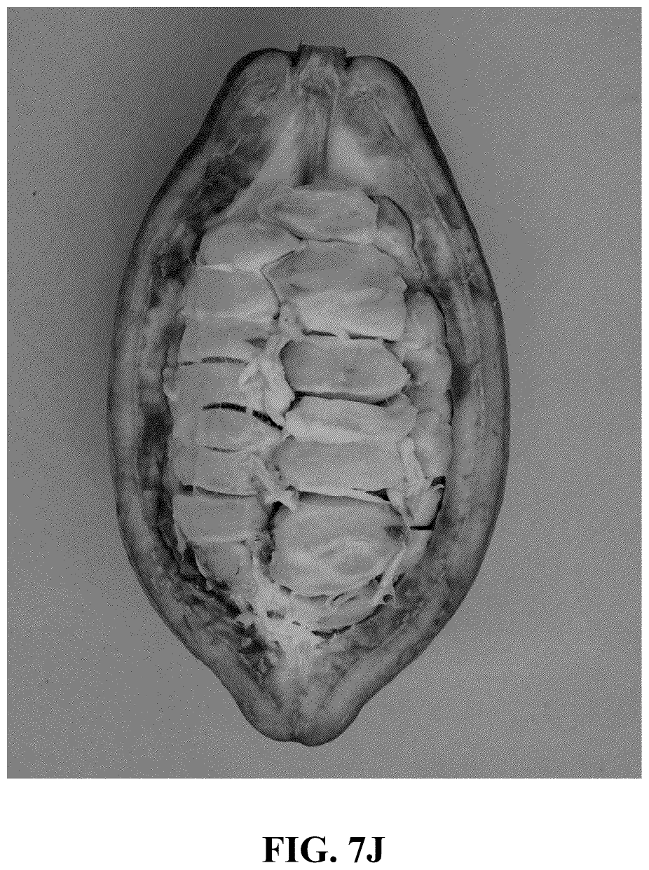

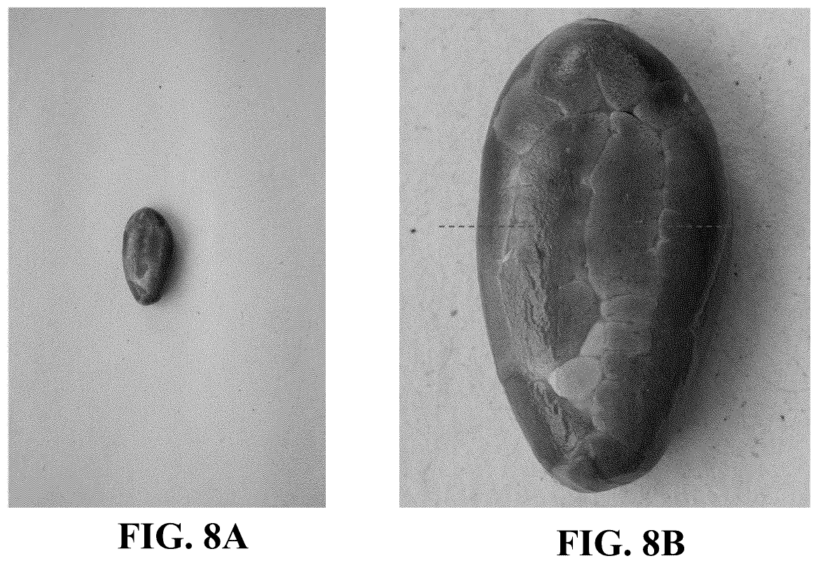

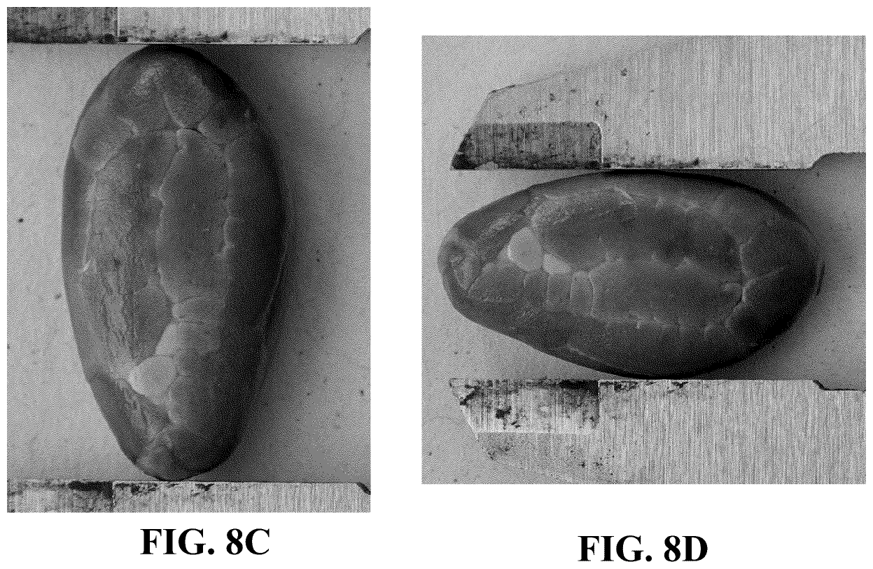

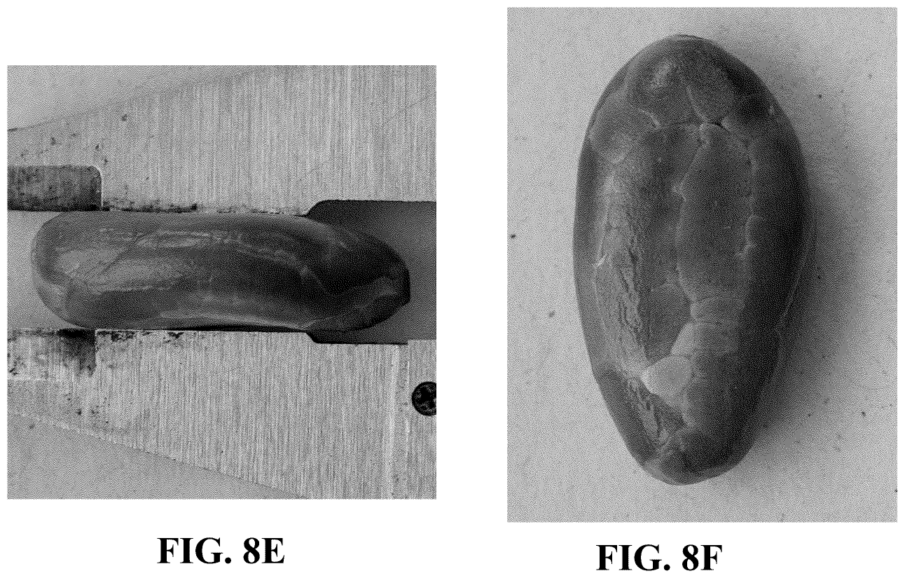

The variety description information in Table 2 provides a summary of variety ‘ISC-2’ plant characteristics. Those of skill in the art will recognize that these are typical values that may vary due to environment, and that other values that are substantially equivalent are within the scope of the invention. The terminology and descriptors are in line with the descriptors of the “UPOV Guidelines for the Conduct of Tests for Distinctness, Uniformity, and Stability”, or the “Test Guidelines” for Theobroma cacao L. The “Test Guidelines” indicate reference varieties for the descriptors or characteristics that are included in the list. The terminology and descriptors used in these tables are in line with the official terminology as of the filing date, and are thus clear for a person skilled in the art. FIGS. 5A-8F illustrate the plant characteristics of variety ‘ISC-2’. FIGS. 18A-18G illustrate the success of the methods used to produce self-compatible variety ‘ISC-2’. FIG. 19A and FIG. 19C illustrate additional data demonstrating the superior yield of variety ‘ISC-2’ across crop years (shown in light purple and labeled “44|GTB11C18R33|MCC02×S1”). FIG. 20B illustrates the progress testing survival rate data for variety ‘ISC-2’.

The following detailed descriptions set forth the distinctive characteristics of ‘ISC-2’. The data which define these characteristics is based on observations taken in Pangkep, Indonesia. This description is in accordance with UPOV terminology. Color designations, color descriptions, and other phenotypical descriptions may deviate from the stated values and descriptions depending upon variation in environmental, seasonal, climatic, and cultural conditions. ‘ISC-2’ has not been observed under all possible environmental conditions. The indicated values represent averages calculated from measurements of several plants. Color references are primarily to The R.H.S. Colour Chart of The Royal Horticultural Society of London (R.H.S.) (2015 edition). Descriptive terminology follows the Plant Identification Terminology, An Illustrated Glossary, 2^{nd} edition by James G. Harris and Melinda Woolf Harris, unless where otherwise defined.

The cacao variety ‘ISC-2’ originated from a controlled cross between varieties ‘MCC02’and ‘S1’. The breeder's reference name of variety ‘ISC-2’ is ‘GTB11C18R33’. Cacao variety ‘ISC-2’ has subsequently been asexually propagated via grafting onto rootstock.
Classification:
   Species.—Theobroma cacao L.
   Common name.—Cacao.
   Denomination.—‘ISC-2’.
Parentage:
   Female parent.—‘MCC02’ (unpatented).
   Male parent.—‘S1’ (unpatented).

TABLE 2
‘ISC-2’.

| Characteristic | Value |
|---|---|
| Leaf blade: length | 29 cm |
| Leaf blade: width | 11.1 cm |
| Leaf blade: shape of base | Obtuse |
| Leaf blade: intensity of green color | Dark |
| Leaf blade: apex | Acuminate |
| Young leaf: color - soon | Brown |
| Young leaf: color - early | Dark red |
| Young leaf: color - medium | Medium green |
| Flower: anthocyanin coloration of pedicel | Strong |
| Flower: length of sepal | 0.8 cm |
| Flower: width of sepal | 0.2 cm |
| Flower: anthocyanin coloration of sepal | Moderate |
| Flower: color of ligula | Yellow |
| Staminode: anthocyanin coloration | Medium |
| Fruit: shape | Elliptic |
| Fruit: basal constriction | Weak |
| Fruit: shape of apex | Obtuse |
| Fruit: length | 16.86 cm |
| Fruit: diameter | 9.61 cm |
| Fruit: length/diameter ratio | 1.75:1 |
| Fruit: surface | Smooth/slightly rough |
| Fruit: depth of groove | Very shallow |
| Fruit: color | Yellow |
| Fruit: exocarp thickness | Medium; 1.55 cm |
| Fruit: color of pulp | White |
| Fruit: sweetness of pulp | μ15.34° Brix |
| Fruit: number of seeds | Pod 1: 41 seeds |
| | Pod 2: 44 seeds |
| Seed: shape in longitudinal section | Elliptic |
| Seed: length | 3.04 cm |
| Seed: width | 1.55 cm |
| Seed: ratio length/width | 1.96:1 |
| Seed: thickness | 0.98 cm |
| Seed: cotyledon color | Purple |

The self-compatible phenotype of cacao variety ‘ISC-2’ has been confirmed through field observation by performing controlled (hand) self-pollinations.

### Variety ‘ISC-3’

In certain aspects, the present invention relates to a new and distinctive cacao plant designated as ‘ISC-3’. Cacao variety ‘ISC-3’ has shown uniformity and stability for the traits, within the limits of environmental influence for the traits. Cacao variety ‘ISC-3’ has been increased with continued observation for uniformity. No variant traits have been observed or are expected in ‘ISC-3’.

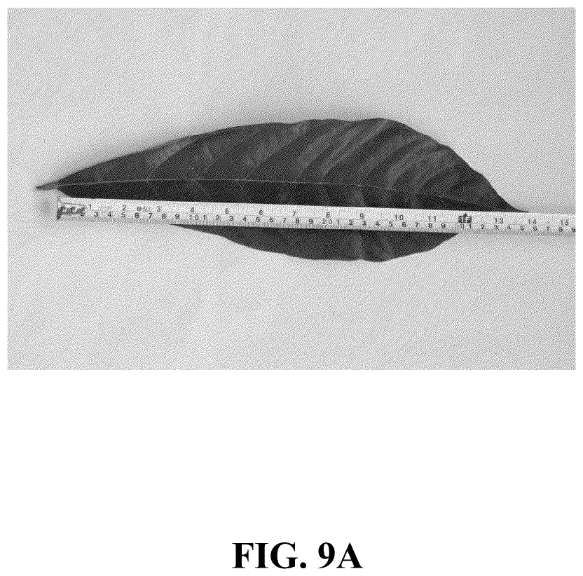

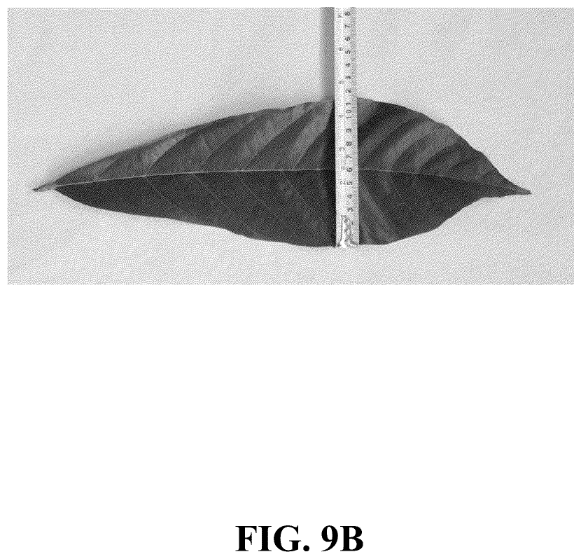

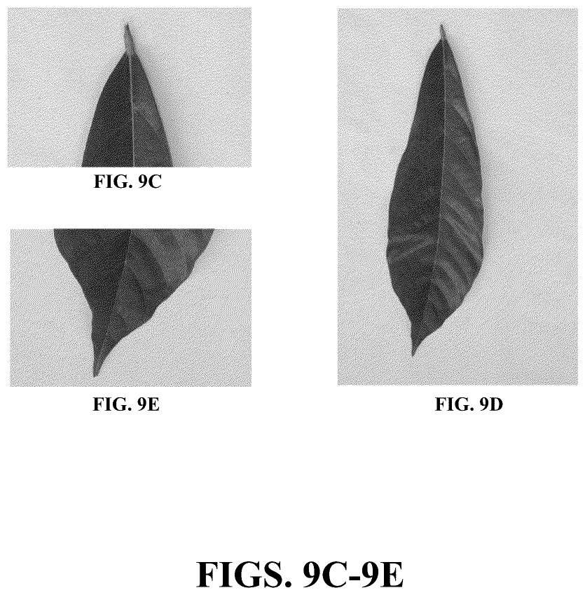

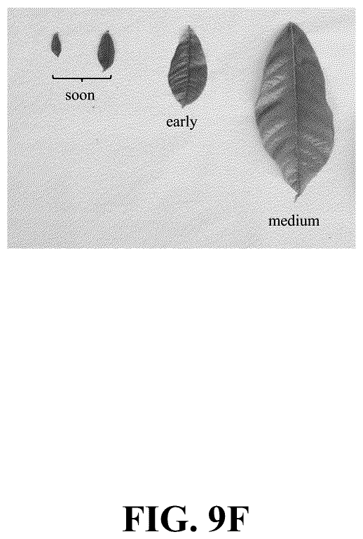

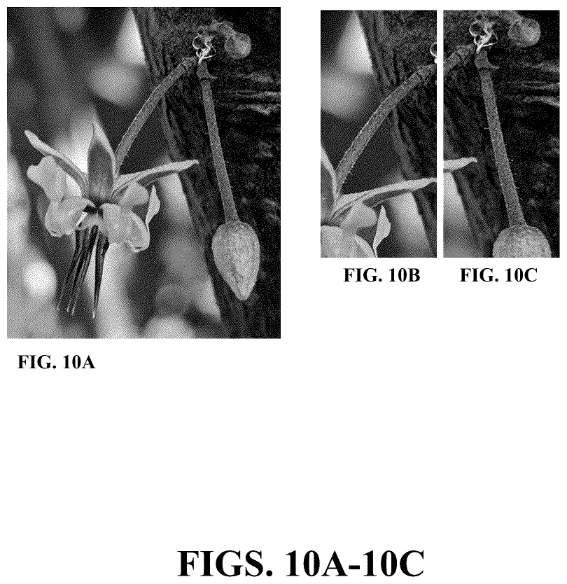

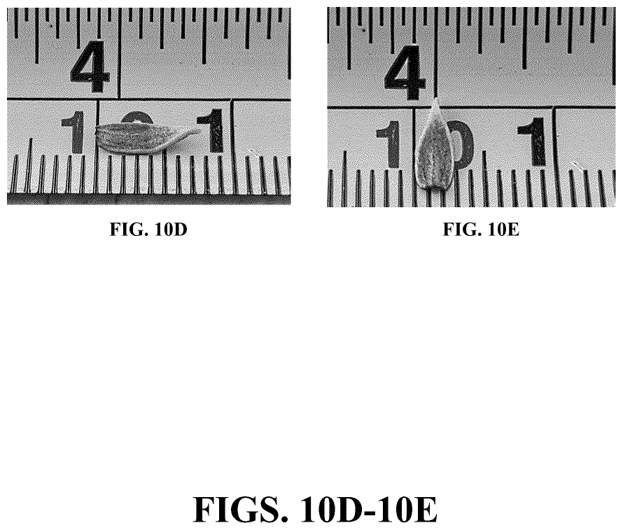

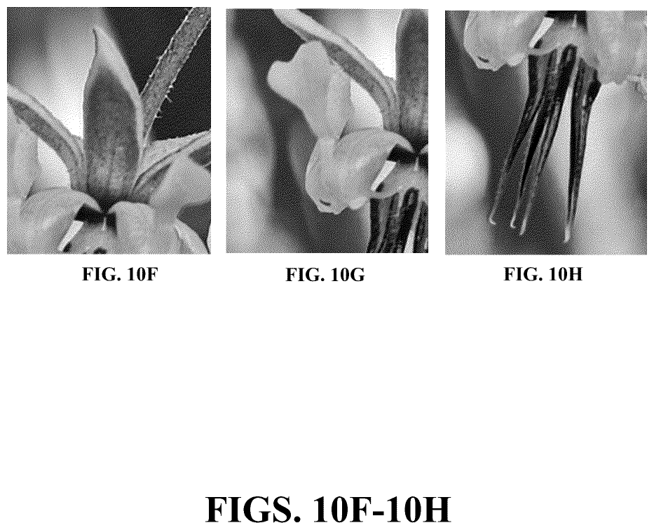

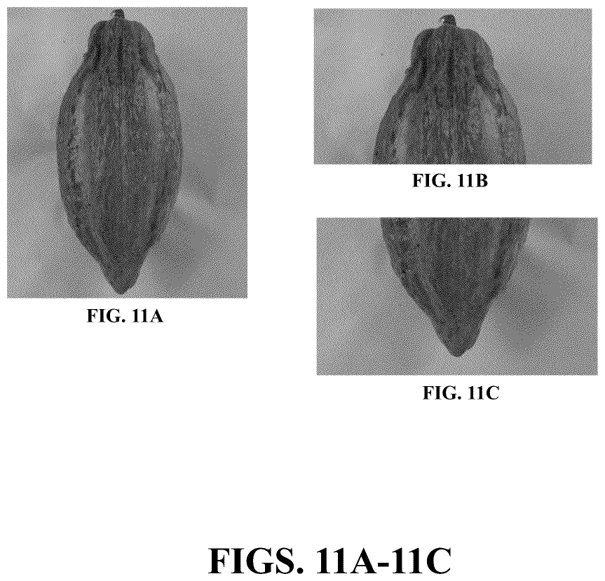

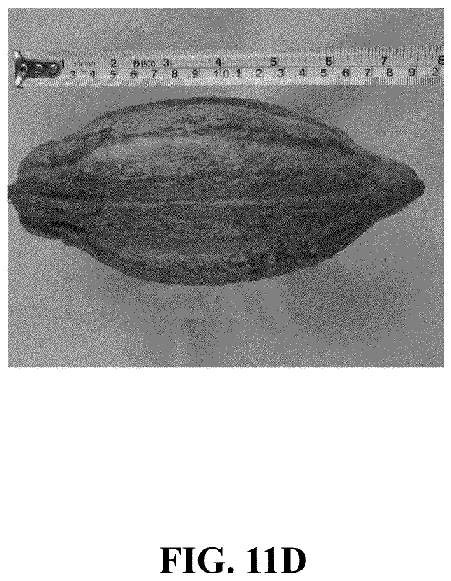

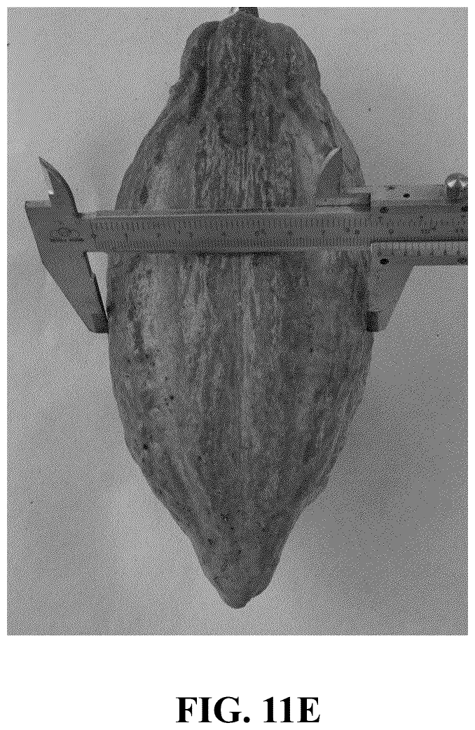

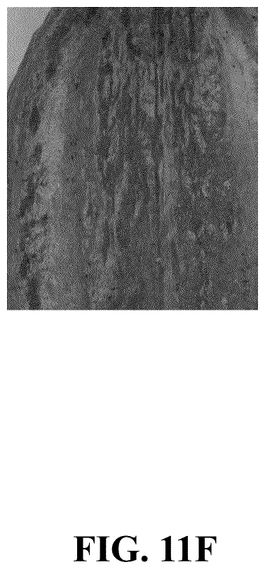

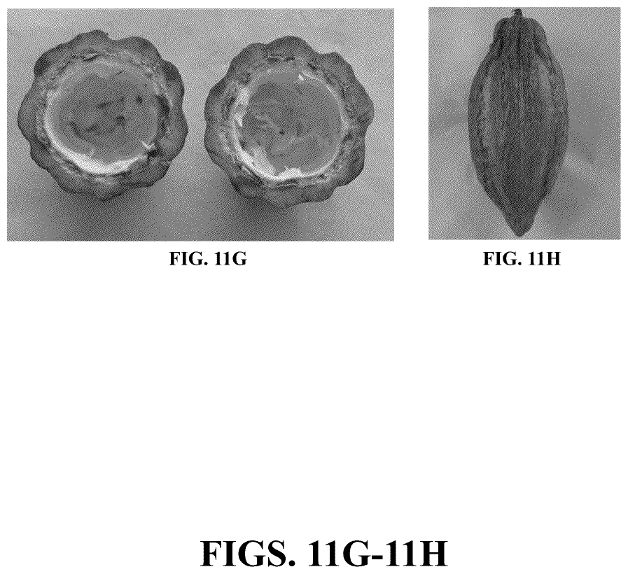

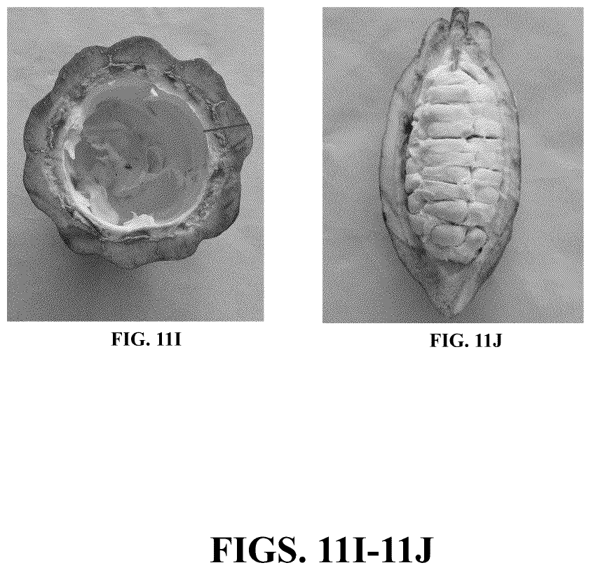

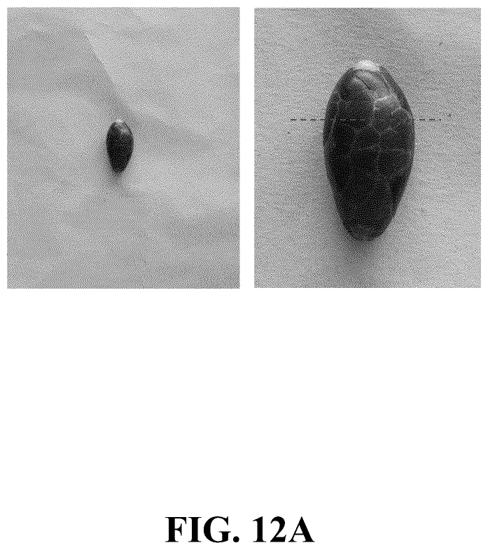

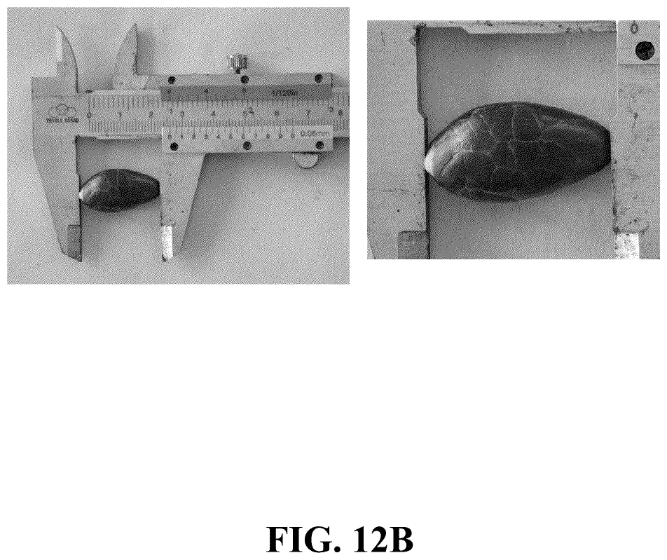

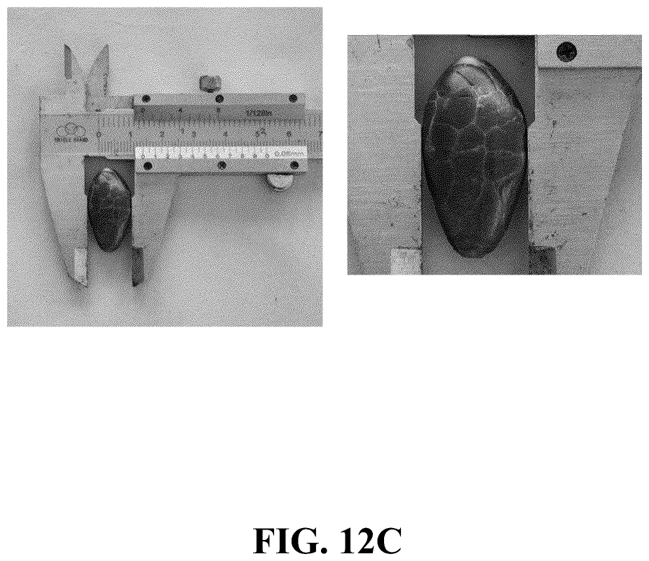

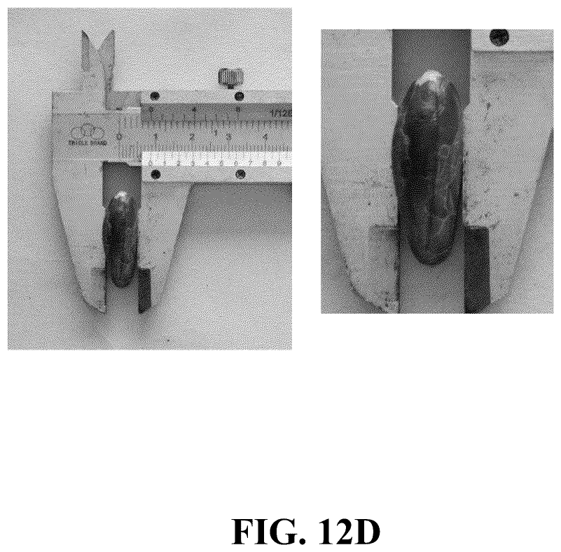

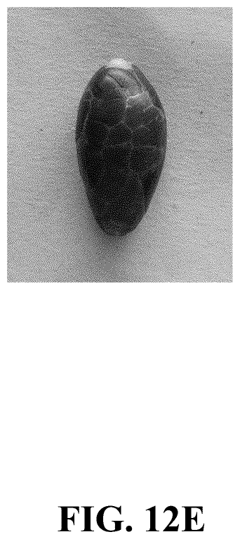

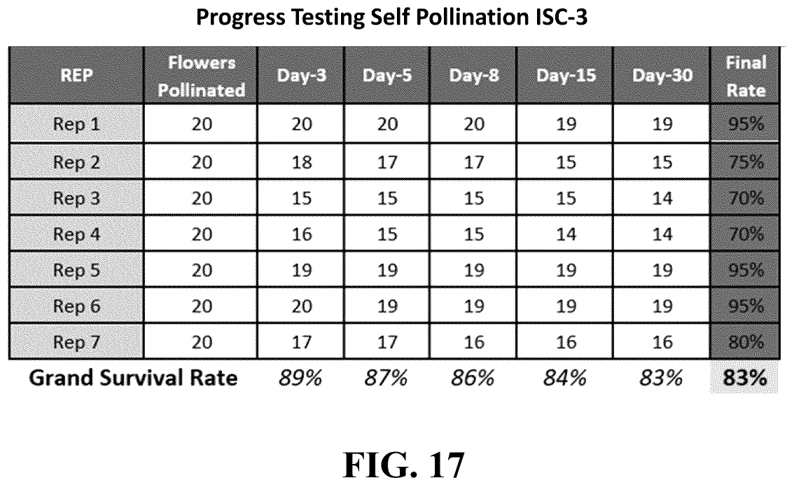

The variety description information in Table 3 provides a summary of variety ‘ISC-3’ plant characteristics. Those of skill in the art will recognize that these are typical values that may vary due to environment, and that other values that are substantially equivalent are within the scope of the invention. The terminology and descriptors are in line with the descriptors of the “UPOV Guidelines for the Conduct of Tests for Distinctness, Uniformity, and Stability”, or the “Test Guidelines” for Theobroma cacao L. The “Test Guidelines” indicate reference varieties for the descriptors or characteristics that are included in the list. The terminology and descriptors used in these tables are in line with the official terminology as of the filing date, and are thus clear for a person skilled in the art. FIGS. 9A-12E illustrate the plant characteristics of variety ‘ISC-3’, and FIG. 17 illustrates the high long-term survival rate of self-pollinated ‘ISC-3’ flowers, which is indicative of very successful self-fertilization. FIGS. 18A-18G illustrate the success of the methods used to produce self-compatible variety ‘ISC-3’. FIG. 19A and FIG. 19C illustrate additional data demonstrating the superior yield of variety ‘ISC-3’ across crop years (shown in green and labeled “5|GTB02C38R21|MCC02×RUQ1347”).

The following detailed descriptions set forth the distinctive characteristics of ‘ISC-3’. The data which define these characteristics is based on observations taken in Pangkep, Indonesia. This description is in accordance with UPOV terminology. Color designations, color descriptions, and other phenotypical descriptions may deviate from the stated values and descriptions depending upon variation in environmental, seasonal, climatic, and cultural conditions. ‘ISC-3’ has not been observed under all possible environmental conditions. The indicated values represent averages calculated from measurements of several plants. Color references are primarily to The R.H.S. Colour Chart of The Royal Horticultural Society of London (R.H.S.) (2015 edition). Descriptive terminology follows the Plant Identification Terminology, An Illustrated Glossary, 2nd edition by James G. Harris and Melinda Woolf Harris, unless where otherwise defined.

The cacao variety ‘ISC-3’ originated from a controlled cross between varieties ‘MCC02’ and ‘RUQ1347’. The breeder's reference name of variety ‘ISC-3’ is ‘GTB02C38R21’. Cacao variety ‘ISC-3’ has subsequently been asexually propagated via grafting onto rootstock.
Classification:
   Species.—Theobroma cacao L.
   Common name.—Cacao.
   Denomination.—‘ISC-3’.
Parentage:
   Female parent.—‘MCC02’ (unpatented).
   Male parent.—‘RUQ1347’ (unpatented).

TABLE 3
‘ISC-3’.

| Characteristic | Value |
|---|---|
| Leaf blade: length | 30.4 cm |
| Leaf blade: width | 11.3 cm |
| Leaf blade: shape of base | Acute |
| Leaf blade: intensity of green color | Dark |
| Leaf blade: apex | Acuminate |
| Young leaf: color - soon | Brown |
| Young leaf: color - early | Brown |
| Young leaf: color - medium | Medium green |
| Flower: anthocyanin coloration of pedicel | Strong |
| Flower: length of sepal | 0.75 cm |
| Flower: width of sepal | 0.25 cm |
| Flower: anthocyanin coloration of sepal | Moderate |
| Flower: color of ligula | Yellow |
| Staminode: anthocyanin coloration | Strong |
| Fruit: shape | Elliptic |
| Fruit: basal constriction | Moderate |
| Fruit: shape of apex | Acute |
| Fruit: length | 19.3 cm |
| Fruit: diameter | 8.68 cm |
| Fruit: length/diameter ratio | 1.98:1 |
| Fruit: surface | Moderately rough |
| Fruit: depth of grooves | Medium |
| Fruit: color | Medium red |
| Fruit: exocarp thickness | Medium thickness: 1.66 cm |
| Fruit: color of pulp | White |
| Fruit: sweetness of pulp | Freshness: μ15.92° Brix |
| Fruit: number of seeds | Pod 1: 41 |
| | Pod 2: 38 |
| Seed: shape in longitudinal section | Elliptic |
| Seed: length | 2.6 cm |
| Seed: width | 1.39 cm |
| Seed: ratio length/width | 1.81:1 |
| Seed: thickness | 0.98 cm |
| Seed: cotyledon color | Dark purple |

The self-compatible phenotype of cacao variety ‘ISC-3’ has been confirmed through field observation by performing controlled (hand) self-pollinations.

### COMPARISON WITH PARENTAL AND REFERENCE VARIETIES

Cacao variety ‘MCC02’ is the industry standard non-selfing clone. It may be used as a comparison variety for each of the self-compatible clones ‘ISC-1’, ‘ISC-2’, and ‘ISC-3’.

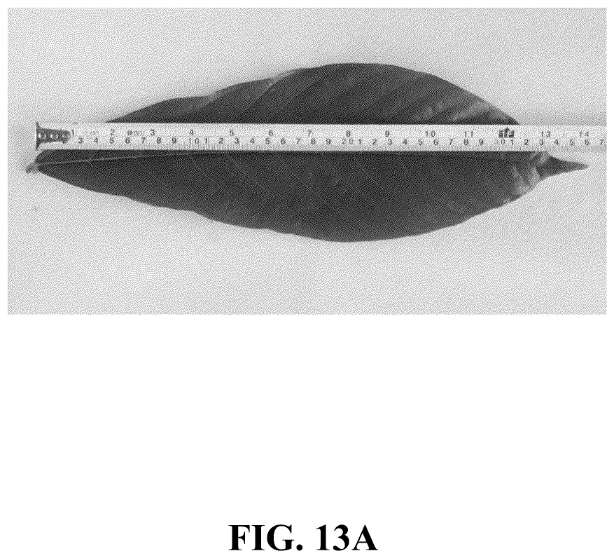

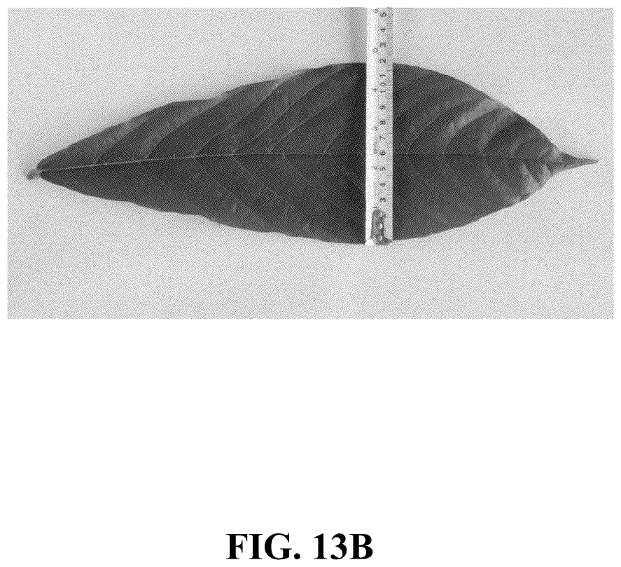

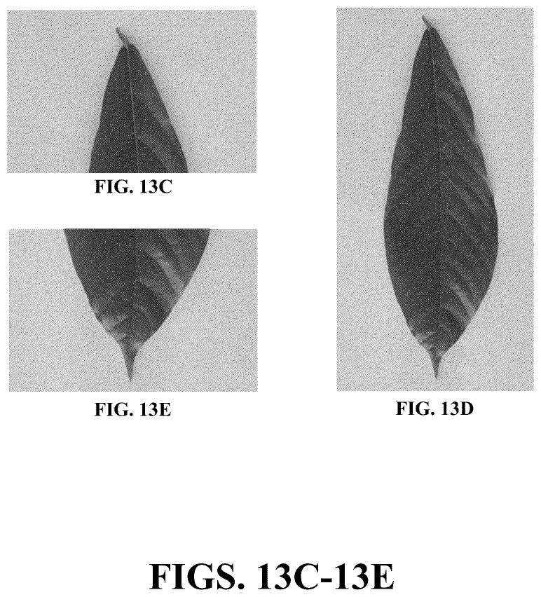

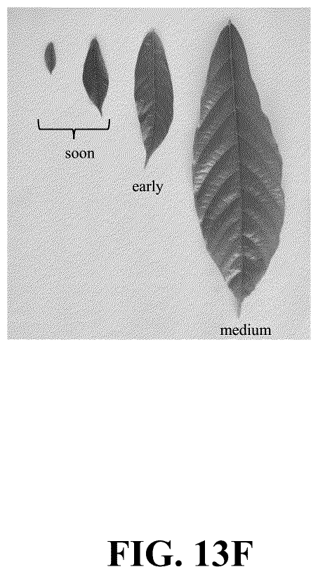

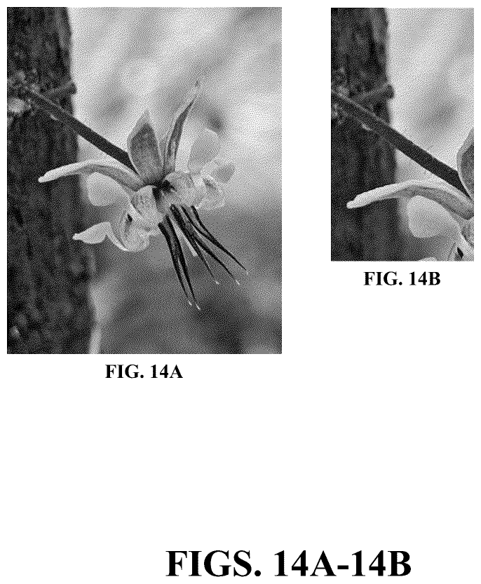

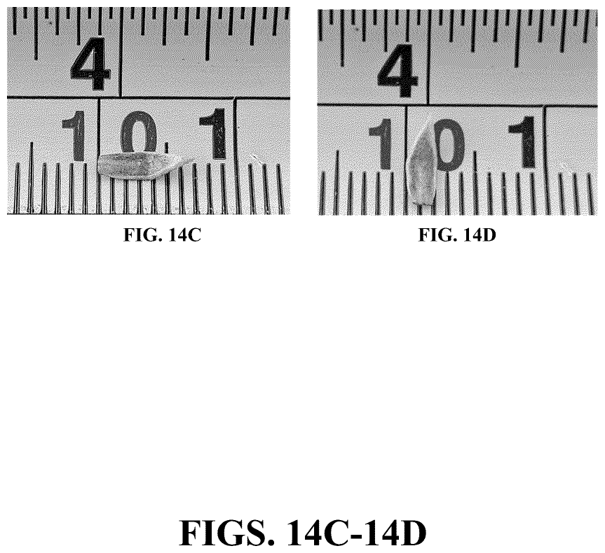

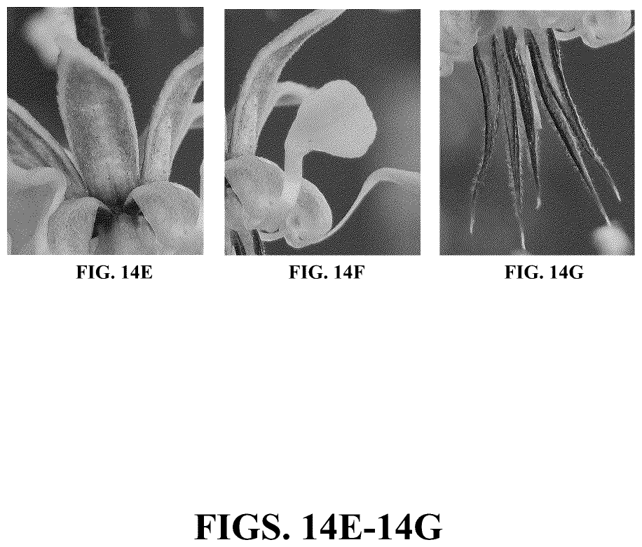

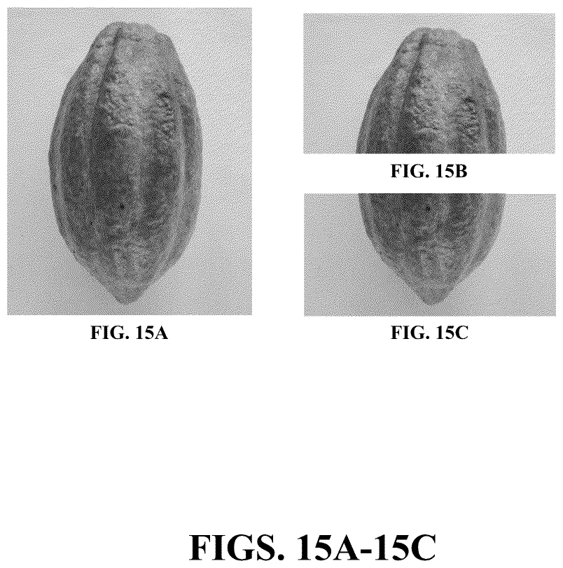

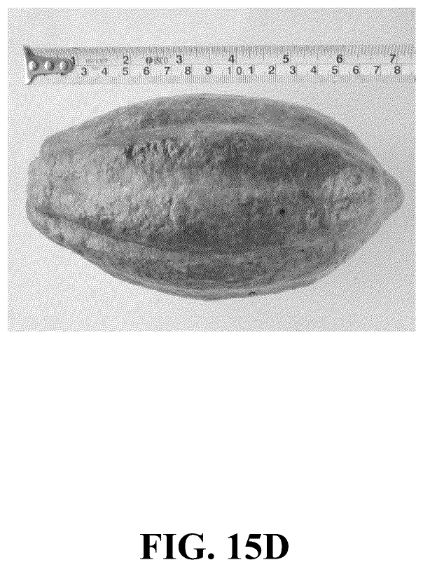

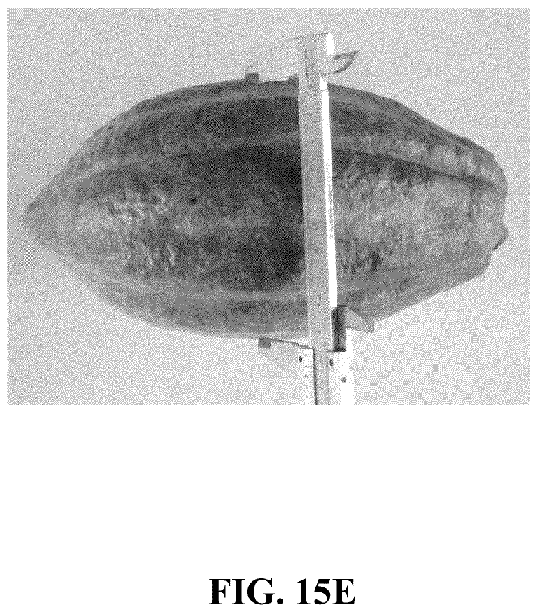

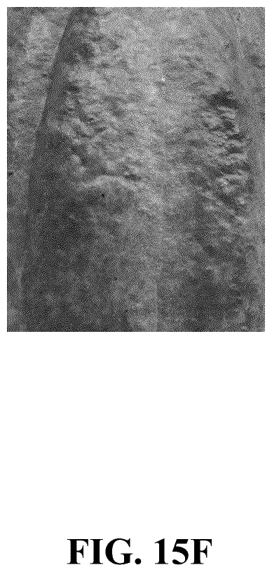

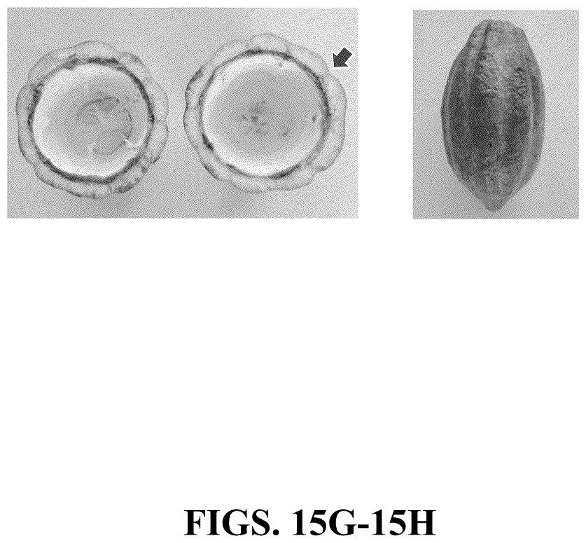

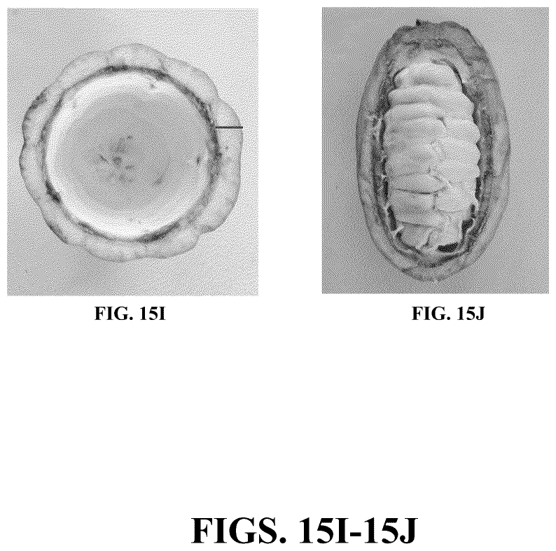

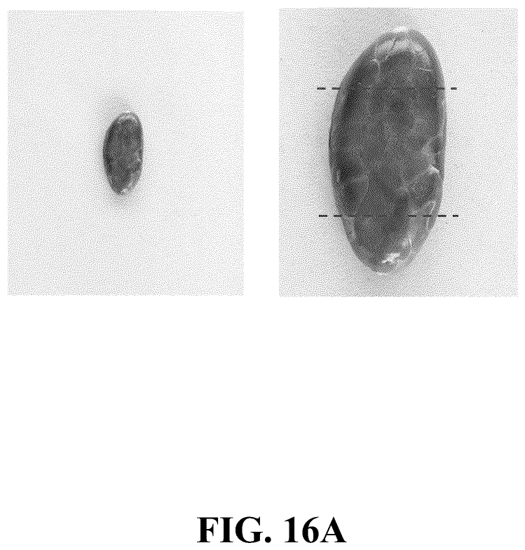

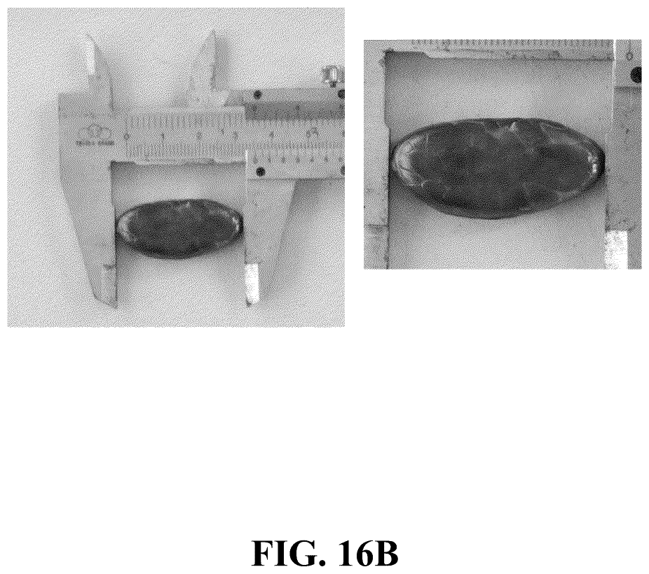

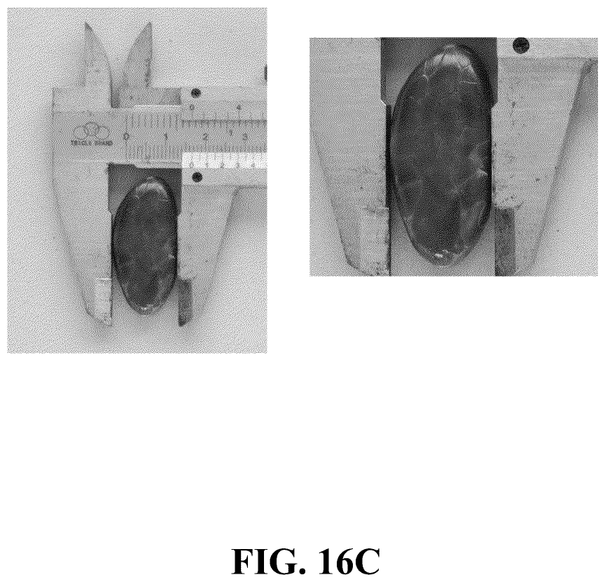

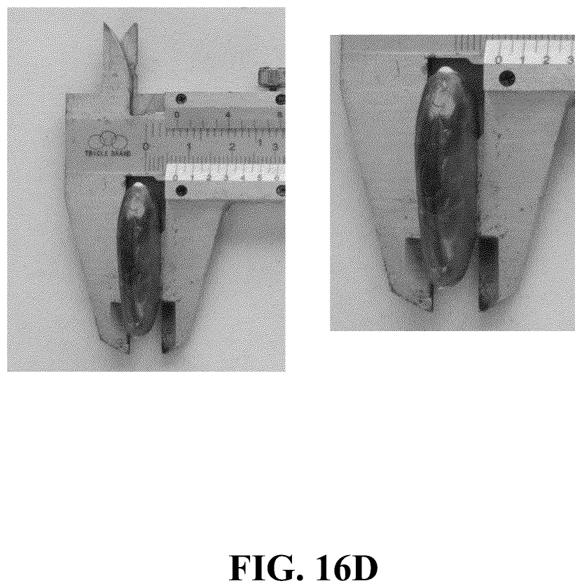

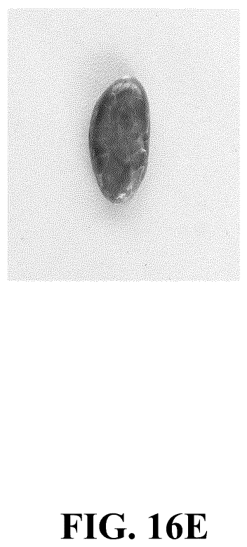

The variety description information in Table 4 provides a summary of comparison variety ‘MCC02’ plant characteristics. Those of skill in the art will recognize that these are typical values that may vary due to environment, and that other values that are substantially equivalent are within the scope of the invention. The terminology and descriptors are in line with the descriptors of the “UPOV Guidelines for the Conduct of Tests for Distinctness, Uniformity, and Stability”, or the “Test Guidelines” for Theobroma cacao L. The “Test Guidelines” indicate reference varieties for the descriptors or characteristics that are included in the list. The terminology and descriptors used in these tables are in line with the official terminology as of the filing date and are thus clear for a person skilled in the art. FIGS. 13A-16E illustrate the plant characteristics of comparison variety ‘MCC02’.

TABLE 4
Comparison variety ‘MCC02’

| Characteristic | Value |
|---|---|
| Leaf blade: length | 35.9 cm |
| Leaf blade: width | 11.7 cm |
| Leaf blade: shape of base | Acute |
| Leaf blade: intensity of green color | Dark |
| Leaf blade: apex | Acuminate |
| Young leaf: color - soon | Brown-medium red |
| Young leaf: color - early | Light red |
| Young leaf: color - medium | Medium green |
| Flower: anthocyanin coloration of pedicel | Strong |
| Flower: length of sepal | 0.7 cm |
| Flower: width of sepal | 0.2 cm |
| Flower: anthocyanin coloration of sepal | Moderate |
| Flower: color of ligula | Yellow |
| Staminode: anthocyanin coloration | Strong |
| Fruit: shape | Obovate |
| Fruit: basal constriction | Weak |
| Fruit: shape of apex | Obtuse |
| Fruit: length | 18 cm |
| Fruit: diameter | 9.92 cm |
| Fruit: length/diameter ratio | 1.81:1 |
| Fruit: surface | Slightly rough |
| Fruit: depth of grooves | Shallow |
| Fruit: color | Medium red-orange |
| Fruit: exocarp thickness | Medium thickness: 1.94 |
| Fruit: color of pulp | White |
| Fruit: sweetness of pulp | Freshness: μ17.14° Brix |
| Fruit: number of seeds | Pod 1: 45 beans |
| | Pod 2: 45 beans |
| Seed: shape in longitudinal section | Oblong |
| Seed: length | 3.55 cm |
| Seed: width | 1.66 cm |
| Seed: ratio length/width | 2.14:1 |
| Seed: thickness | 1.12 cm |
| Seed: cotyledon color | Purple |

‘ISC-1’ differs from the female parent cacao plant and reference cacao variety ‘MCC02’ (unpatented) in that ‘ISC-1’ has improved yield compared to ‘MCC02’, and in that ‘ISC-1’ is self-compatible while ‘MCC02’ is non-selfing.

‘ISC-2’ differs from the female parent cacao plant and reference cacao variety ‘MCC02’ (unpatented) in that ‘ISC-2’ has improved yield compared to ‘MCC02’, and in that ‘ISC-2’ is self-compatible while ‘MCC02’ is non-selfing.

‘ISC-3’ differs from the female parent cacao plant and reference cacao variety ‘MCC02’ (unpatented) in that ‘ISC-3’ has improved yield compared to ‘MCC02’, and in that ‘ISC-3’ is self-compatible while ‘MCC02’ is non-selfing.

### REFERENCES

Glendinning D R. 1967. Incompatibility alleles of cocoa. Nature 213, 306.
Marshall J (1933) Fertility in cacao. 3rd Annual Report on Cacao Research (Trinidad), p 34
Pound. F. J. “Studies of fruitfulness in cacao. I. A note on the abscission of the flower. II. Evidence for partial sterility.” Studies of fruitfulness in cacao. I. A note on the abscission of the flower. II. Evidence for partial sterility. (1932).

We claim:

1. A new and distinct variety of cacao plant as shown and described herein, wherein the variety is selected from the group consisting of ‘ISC-1’, ‘ISC-2’, and ‘ISC-3’.
